(19) United States
(12) Patent Application Publication
(10) Pub. No.: US 2026/0009800 A1
(43) Pub. Date: Jan. 8, 2026

(54) METHODS OF DIAGNOSIS AND GUIDING TREATMENT OF VULVOVAGINAL SYMPTOMS AND FEMALE SEXUAL DYSFUNCTION

(71) Applicant: ARIZONA BOARD OF REGENTS ON BEHALF OF THE UNIVERSITY OF ARIZONA, Tucson, AZ (US)

(72) Inventors: Melissa M. Herbst-Kralovetz, Phoenix, AZ (US); Pawel Laniewski, Phoenix, AZ (US)

(21) Appl. No.: 19/325,243

(22) Filed: Sep. 10, 2025

Related U.S. Application Data
(63) Continuation-in-part of application No. PCT/US2024/019447, filed on Mar. 11, 2024.
(60) Provisional application No. 63/489,641, filed on Mar. 10, 2023.

(51) Int. Cl. G01N 33/68; A61K 38/22; A61K 45/06; A61P 15/02

(52) U.S. Cl. CPC G01N 33/68; A61K 38/22; A61K 45/06; A61P 15/02; G01N 2405/08; G01N 2800/36; G01N 2800/60

(57) ABSTRACT

Sexual dysfunction, including symptoms of vaginal dryness, irritation, and dyspareunia, is a highly prevalent condition with a substantial impact on women's physical, mental, and social well-being. However, the underlying mechanisms responsible for these symptoms are largely unknown, often resulting in inadequate treatment options for patients. Thus, methods to determine interactions between vaginal microbiota, metabolites, and immune proteins in women with or without these symptoms are described herein. The objective of these methods is to improve the understanding of the pathologic mechanisms contributing to poor sexual health.

### CROSS-REFERENCE TO RELATED APPLICATIONS

This application is a continuation-in-part and claims benefit of International Application No. PCT/US2024/019447 filed Mar. 11, 2024, which claims benefit of U.S. Provisional Application No. 63/489,641 filed Mar. 10, 2023, the specifications of which are incorporated herein in their entirety by reference.

### FIELD OF THE INVENTION

The present invention features methods for a non-invasive test for guiding treatments of vulvovaginal symptoms and female sexual dysfunction. Methods described herein may be used in both pre- and postmenopausal women suffering from vulvar and vaginal symptoms, such as dryness, irritation, painful intercourse, pain during urination, etc.

### BACKGROUND OF THE INVENTION

Women during and after menopause frequently suffer from genitourinary symptoms, such as vulvovaginal atrophy, dyspareunia, dysuria, and recurrent urinary tract infections. Although these symptoms usually improve over time, the genitourinary syndrome of menopause (GSM) usually worsens without effective treatment. The vulvovaginal and urinary symptoms can also occur in premenopausal women, especially in women with benign gynecologic conditions, such as endometriosis, adenomyosis, and fibroids. Importantly, these symptoms can significantly impair women's sexual function, satisfaction, and overall quality of life. Yet, multiple physical, hormonal, and psychological factors can contribute to female sexual dysfunction.

Furthermore, managing vulvovaginal symptoms and female sexual dysfunction can vary from non-medical treatments (such as lifestyle and home remedies, counseling, and lubricant use) to medical treatments (such as hormone therapies: estrogen, estrogen receptor, or androgen therapies). However, no test is available to guide a physician in choosing the optimal treatment for a woman with particular genitourinary symptoms. Thus, the present invention features a multi-omics approach combined with cervicovaginal sampling that allows the identification of specific immune mediators, metabolites, and bacteria related to vulvovaginal and sexual health symptoms. The pathophysiological mechanisms underlying these symptoms aid in guiding as well as developing treatment options for women undergoing menopause or women with benign gynecological conditions experiencing sexual dysfunction and ultimately improve women's quality of life.

### BRIEF SUMMARY OF THE INVENTION

It is an objective of the present invention to provide methods that allow for a non-invasive test for guiding treatments of vulvovaginal symptoms and female sexual dysfunction. Methods described herein may be used in both pre- and postmenopausal women suffering from vulvar and vaginal symptoms, such as dryness, irritation, painful intercourse, pain during urination, etc., as specified in the independent claims. Embodiments of the invention are given in the dependent claims. Embodiments of the present invention can be freely combined with each other if they are not mutually exclusive.

In some embodiments, the present invention features a method for providing a therapeutic solution to treat vulvovaginal symptoms in a female patient in need thereof. In some embodiments, the method comprises: determining the patient's levels of two or more biomarkers, applying the results to predetermined thresholds, wherein deviation of at least two biomarkers from the thresholds identifies at least one therapeutic solution for treating the vulvovaginal symptoms, and providing the therapeutic solution to a medical professional to guide treatment of the patient. In some embodiments, the levels of two or more biomarkers are determined by obtaining a biological sample from the patient and measuring the levels of two or more biomarkers in the biological sample obtained. In some embodiments, the biological sample comprises a cervicovaginal lavage (CVL) sample or a vaginal swab. In some embodiments, the vulvovaginal symptoms are caused by genitourinary syndrome of menopause (GSM) and may include vaginal dryness, vaginal irritation, vaginal soreness, dyspareunia, vulvar dryness, vulvar irritation, vulvar soreness, and pain. In some embodiments, the method comprises measuring the levels of at least five or more, at least ten or more, or at least twenty or more biomarkers. In some embodiments, the therapeutic solutions comprise one or a combination of hormonal therapies, antimicrobials, anti-inflammatories, neuromodulators, vaginal moisturizers, antihistamines, or pelvic floor therapy.

In some embodiments, the present invention features a method of treating vulvovaginal symptoms in a female patient in need thereof. The method comprises determining the patient's levels of two or more biomarkers and treating the patient based on the levels of said biomarkers. The level of two or more biomarkers may be determined by obtaining a cervicovaginal lavage (CVL) sample from the patient and measuring the levels of at least two or more biomarkers in the sample obtained.

In some embodiments, the biomarkers comprise immune proteins, metabolites, lipids, bacterial abundance, or a combination thereof. In some embodiments, the metabolites comprise amino acids, nucleotides, lipids (such as glycerophospholipids, fatty acids, or sphingolipids), xenobiotics, or a combination thereof.

One of the unique and inventive technical features of the present invention is the use of a non-invasive method to guide the treatment of vulvovaginal symptoms and female sexual dysfunction. As described herein, the invention provides physicians with objective guidance for optimizing patient treatment by integrating a patient's verbal description of symptoms with biomarker-based measurements that quantify symptom severity. Without wishing to limit the invention to any theory or mechanism, it is believed that this technical feature of the present invention advantageously enables more precise and individualized treatment decisions, including the ability to distinguish between hormonal and non-hormonal treatment options (e.g., estrogen versus non-drug interventions) based on the presence, absence, or levels of particular immunometabolic and microbial biomarkers. In this way, the invention not only enhances treatment decisions but may also help reduce medical bias, such as the underestimation of symptoms based solely on patient self-reporting. None of the presently known prior references or work disclose this unique and inventive technical feature of the present invention.

Non-limiting examples of how the methods described herein may guide treatment include the following. In certain embodiments, at least one of the two or more biomarkers comprises histamine. In other embodiments, the two or more biomarkers comprise histamine and at least one carnitine or a derivative thereof, wherein the carnitine derivative is selected from the group consisting of laurylcarnitine (C12), 5-dodecenoylcarnitine, myristoleoylcarnitine (C14:1), and decanoylcarnitine (C10). In some embodiments, when histamine is increased relative to a predetermined threshold and the at least one carnitine or derivative is decreased relative to a predetermined threshold, the therapeutic solution comprises an antihistamine. In other embodiments, when histamine is not elevated relative to a predetermined threshold and the levels of carnitine or derivatives thereof are not decreased, the biomarker profile is more consistent with genitourinary syndrome of menopause (GSM). In such embodiments, the therapeutic solution comprises an estrogen therapy, a selective estrogen receptor modulator (SERM), or dehydroepiandrosterone (DHEA)

Furthermore, the inventive technical features of the present invention contributed to a surprising result. For example, the Inventors expected that inflammatory biomarkers would play a bigger role and be a part of the overall profile; however, a completely different set of biomarkers related to immune checkpoint proteins was observed. Additionally, a down-regulation instead of an upregulation of lipids in the metabolomics analysis was surprisingly detected.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A shows Vulvovaginal discomfort measurements strongly positively correlated and negatively correlated with positive sexual health measurements. Correlation matrix of the 8 PROMIS SexFS variables and total VAS and VuAS Scores among all patients; maximum patient numbers were used for each pairwise correlation. Unsupervised hierarchical clustering revealed two distinct clusters. Clustering was based on Euclidean distance and ward linkage. Correlation coefficients (p) were calculated using Spearman's rank correlation analysis. Red and purple squares represent positive and negative correlations, respectively. Significant correlations (P<0.05) are indicated by black circles.

FIGS. 2A, 2B, and 2C shows Distinct metabolite profiles identified between high and low vulvovaginal symptoms. Principal component analysis (PCA) of cervicovaginal metabolite profiles grouped by VAS (FIG. 2A) and VuAS (FIG. 2B). The first two principal components (PC) are displayed with each point representing an individual sample and color-coded by low and high VAS (n=67), and VuAS (n=69) scores. Box-and-whisker plots represent the distribution of samples along each PC and show the median, interquartile range, and 10th and 90th percentiles, with outliers shown as dots. P values between VAS and VuAS groups were calculated using Welch's t-test. * P<0.05. (FIG. 2C) Hierarchical clustering heatmap displays the top 50 significant metabolites between high and low VAS groups, determined by t-test. Unsupervised clustering of metabolites revealed two distinct clusters. Clustering was based on Euclidean distance and Ward linkage. Samples were grouped by high and low total VAS scores, with VuAS groupings also shown. Metabolites are color-coded by superpathway.

FIGS. 3A, 3B, 3C, 3D, 3E, and 3F shows Significant dysregulation of metabolites, predominantly glycerophospholipids, in women with more severe vaginal symptoms. FIG. 3A shows a bar chart that depicts the number of significantly altered metabolites in high VAS and VuAS groups, color-coded by superpathway. Pie chart shows further categorization of lipid superpathways into subpathways. Gluycerophospholipids included plasmalogens, lysophspholipids, phosphatidylethanolamines (PE), phosphatidylinositols (PI), and phosphatidylserines (PS). FIG. 3B shows scatterplots of the top 5 significantly downregulated and 3 significantly upregulated metabolites in women with high VAS scores. P values shown under each plot. Bar charts depict the number of metabolites significantly (P<0.05) correlated with positive (FIG. 3C) and negative (FIG. 3E) PROMIS SexFS measures, color-coded by superpathway. Significant correlations were determined using Spearman's rank correlation analysis. Venn diagrams of the number of shared and unique metabolites that correlate with the 5 positive (FIG. 3D) and 3 negative (FIG. 3F) PROMIS SexFS measures. Tables indicate most highly shared metabolites and their superpathway (colored dots). The 19 shared metabolites listed in (FIG. 3F) all negatively correlated with vaginal discomfort, vulvar discomfort-clitoral, and vulvar discomfort-labial.

FIGS. 4A and 4B shows Significant dysregulation of fatty acid metabolism in women with high vs low VAS scores. FIG. 4A shows a bar plot of the top 25 significantly dysregulated metabolite pathways between women with high and low VAS scores. Pathways ordered by P value (0.0004<P<0.05) and color-coded by superpathway. Top 10 pathways also significant after FDR correction (q<0.05), indicated by asterisks (*). An additional 9 significant (P<0.05) pathways are not depicted. FIG. 4B shows a bar plot of the one significantly (P<0.05) dysregulated pathway between high and low VuAS scores. Enrichment ratio and significance of enrichment of metabolic pathways were calculated based on the number of metabolites detected within a specific pathway relative to the number of known metabolites in that pathway.

FIGS. 5A, 5B, and 5C shows Levels of immune proteins in cervicovaginal lavages significantly correlated with vulvovaginal and sexual health symptoms. FIG. 5A shows scatterplots of the 6 significantly upregulated and downregulated metabolites in women with high VAS scores. P values shown under each plot. FIG. 5B shows dot plots display the immune proteins significantly (P<0.05) correlated with individual and total VAS and VuAS symptoms. Vulvar pain with external touch had no significant correlations with immune proteins. FIG. 5C shows heatmap displays significantly correlated immune proteins with positive and negative sexual health measures. Significant correlations (P<0.05) are indicated by black dots. Correlation coefficients (p) were determined by Spearman correlation analysis.

FIGS. 6A, 6B, and 6C shows Enrichment of vaginal dysbiosis-associated bacteria and microbial metabolic pathways associated with severe vaginal symptoms. FIG. 6A shows relative abundance of taxa at the genus level between low and high VAS groups. FIG. 6B shows scatterplot depicts the log 2 fold change (LFC) of differentially abundant vaginal taxa between high and low VAS scores, VuAS symptoms and no symptoms, and presence and absence of individual VAS symptoms. Differential abundance was identified using ANCOM-BC. Dots indicate significance at q<0.05 and LFC>1, except for vaginal irritation which was significant at P<0.05 (+). No significant differentially abundant vaginal bacteria were identified for individual vulvar symptoms. FIG. 6C shows a volcano plot of PICRUSt pathway analysis showing the differentially abundant significant metabolic pathways up- and downregulated in women with high VAS groups, shown in red (circle) and blue (square), respectively (P<0.05; LFC>1). Each dot represents a pathway from the MetaCyc database. Grey dots represent non-significant pathways. Two significant differentially abundant metabolic pathways between high and low VuAS scores are not depicted.

FIGS. 7A, 7B, 7C, and 7D shows significant correlations among metabolites, immune proteins, and microbes identified. Heatmaps display significant (P<0.05) correlations between (FIG. 7A) altered metabolites and immune proteins, (FIG. 7B) altered metabolites and vaginal bacteria, and (FIG. 7C) altered immune proteins and vaginal bacteria. Altered metabolites and immune proteins refers to any metabolites or immune proteins that were identified as significant in any previous analyses with the eight PROMIS SexFS measures or VAS and VuAS measures. Only correlations with a coefficient <−0.3 or >0.3 are shown; heatmaps (FIG. 7A) and (FIG. 7C) display only the top 50 strongest correlating metabolites. Correlation coefficients (p) were determined by Spearman correlation analysis. Unsupervised clustering was performed for rows and columns, based on Euclidean distance and Ward linkage. Colored squares next to metabolite names indicate the metabolic superpathway.

FIG. 8 shows a significant correlation of metabolites between t-scores and VAS/VuAS. *Positively correlated with sexual t-scores; negatively correlated with VAS/VuAS.

FIG. 9 shows a significant correlation of metabolites between t-scores and VAS/VuAS. *Positively correlated with sexual t-scores; negatively correlated with VAS/VuAS.

FIGS. 10A and 10B show significantly correlated metabolites shared between T-scores and VAS/VuAS.

FIGS. 11A, 11B, 11C, 11D, and 11E show immune protein signatures correlated with VAS and vaginal symptoms (p<0.05). FIG. 11A shows immune checkpoint proteins, PD-1/PD-L1, LAG-3, CD80, and CD-40. FIG. 11B shows TRAIL was upregulated. FIG. 11C shows cytokines, IL-12, IL-2, IL-7, IL-10, TNF-alpha and stem cell factor (SCF) were upregulated. FIG. 11D shows tumor markers, prostate specific antigen (PSA), Carcinoembryonic antigen (CEA), and CYFRA 21-1. FIG. 11E shows cytokines IL-8, RANTES, and Fractalkine. Note: Bars from top to bottom represent-VAS score, Vaginal Irritation, Vaginal dryness, Vaginal soreness, and Dyspareunia.

FIGS. 12A, 12B, 12C, and 12D show differentially abundance of vaginal taxa. Sneathia amnii was enriched in women with high VAS scores, vaginal irritation, and dyspareunia and is linked to barrier disruption and inflammation. Upregulated secretions of proinflammatory cytokines, including IL-8. Megasphaera lornae was enriched in women with dyspareunia—BV-associated bacteria. Streptococcus agalactiae (group B strep) was enriched in women with vaginal soreness and dyspareunia. Stimulates vaginal epithelial exfoliation and loss of barrier function. Known estrogen degrader (link to vaginal atrophy, dryness, and dyspareunia). Lactobacillus crispatus depletion in women experiencing vaginal soreness.

FIG. 13 shows Sexual Health Survey Response Rates.

FIGS. 14A and 14B show VAS and VuAS data.

FIGS. 15A and 15B show significantly altered immune proteins. LFC threshold of 2.0, p-value threshold of 0.05 (not FDR corrected. 2 proteins were significantly downregulated, and four proteins were significantly upregulated).

FIG. 16 shows VAS and lacto-dominance heatmap.

FIG. 17 shows significantly correlated metabolites with VAS/VuAS.

FIG. 18 shows significantly correlated metabolites with t-scores.

FIG. 19 shows the number of unique and shared significantly correlated metabolites between T-scores and VAS/VuAS scores.

FIG. 20 shows significantly altered metabolites between low and high VAS.

FIG. 21 shows significantly altered metabolites between low and high VuAS.

FIG. 22 shows significantly correlated metabolites with sexual satisfaction.

FIG. 23 shows significantly correlated metabolites with sexual interest.

FIG. 24 shows significantly correlated metabolites with orgasm ability.

FIG. 25 shows significantly correlated metabolites with orgasm pleasure.

FIG. 26 shows significantly correlated metabolites with vaginal lubrication.

FIG. 27 shows significantly correlated metabolites with vulvar discomfort-labial.

FIG. 28 shows significantly correlated metabolites with vulvar discomfort-clitoral.

FIG. 29 shows significantly correlated metabolites with vaginal discomfort.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, “immune mediators” may refer to a group of proteins that regulate the immune system. In some embodiments, the level of these proteins can be measured using antibody-based protein assays such as but not limited to ELISA and cytometric bead array. In some embodiments, cervicovaginal lavage (CVL) samples are analyzed using antibody-based protein assays to detect and measure immune checkpoint biomarker proteins.

As used herein, “inflammation” may refer to a localized physical condition in which part of the body becomes reddened and swollen, especially as a reaction to injury or infection or a state of locally elevated levels of pro-inflammatory cytokines and chemokines. In some embodiments, inflammation may be short-lived (acute) or long-lived (chronic).

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human.

The terms “treating” or “treatment” refers to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread or worsening of a disease or disorder or of one or more symptoms thereof. In certain cases, the beneficial effects that a subject derives from a prophylactic or therapeutic agent do not result in a cure of the disease or disorder.

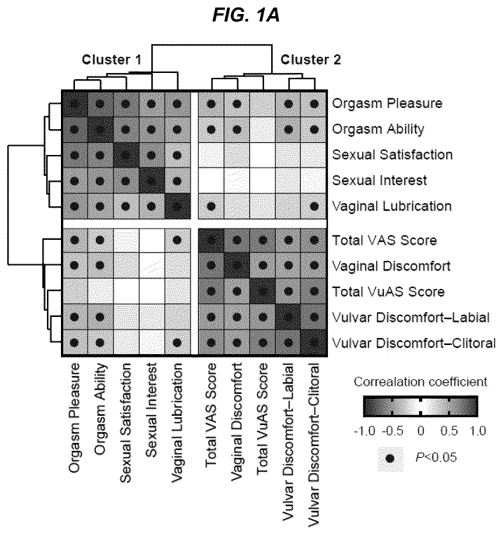

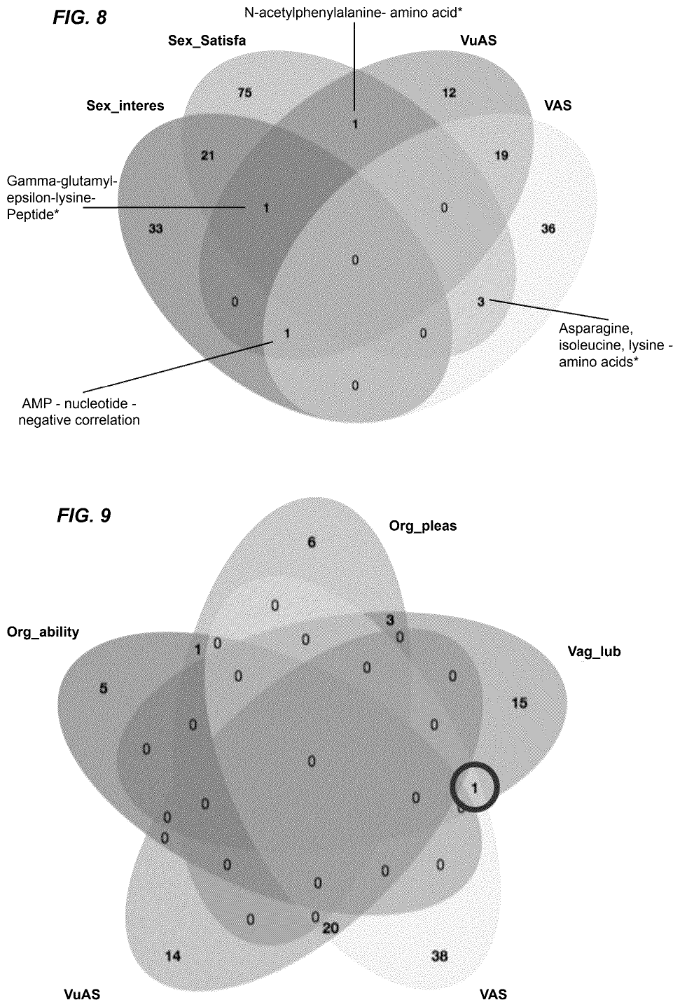

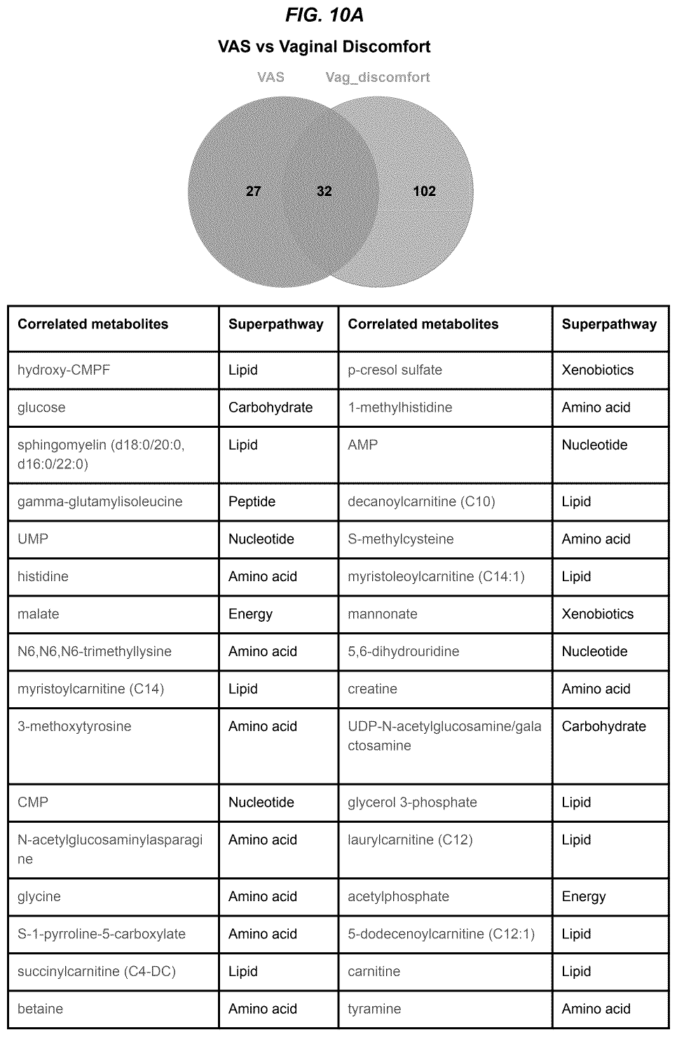

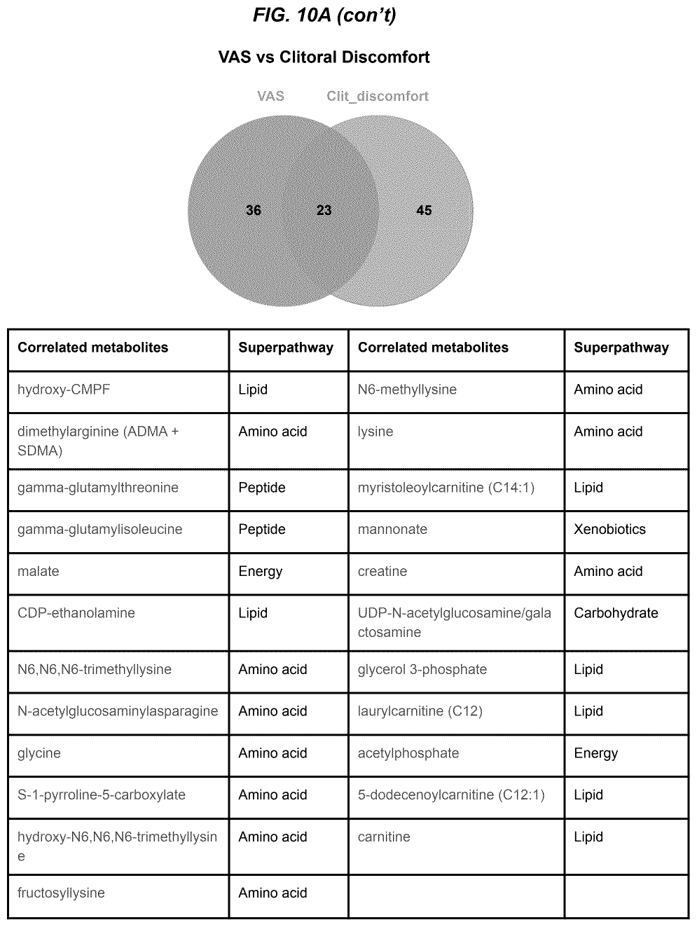

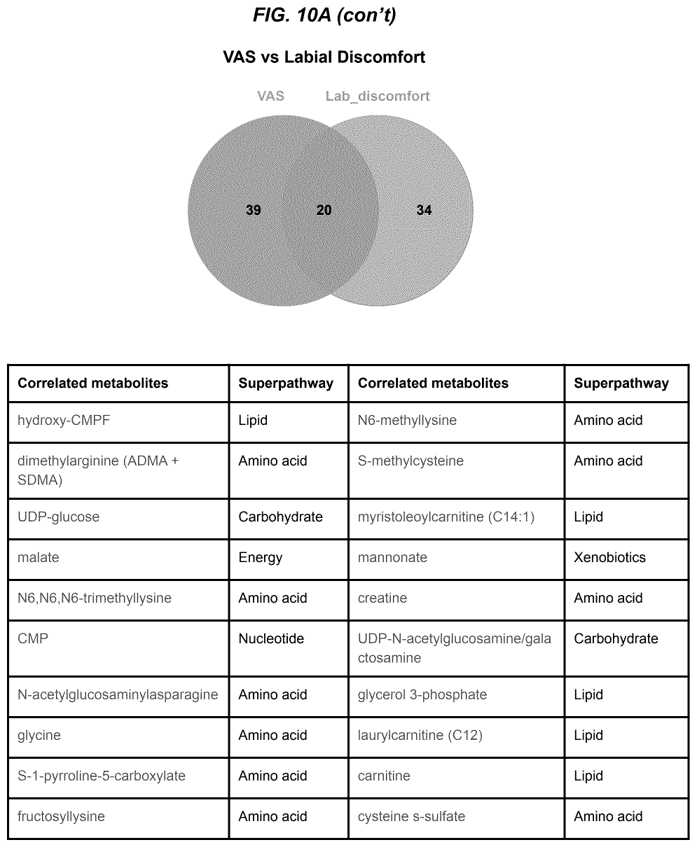

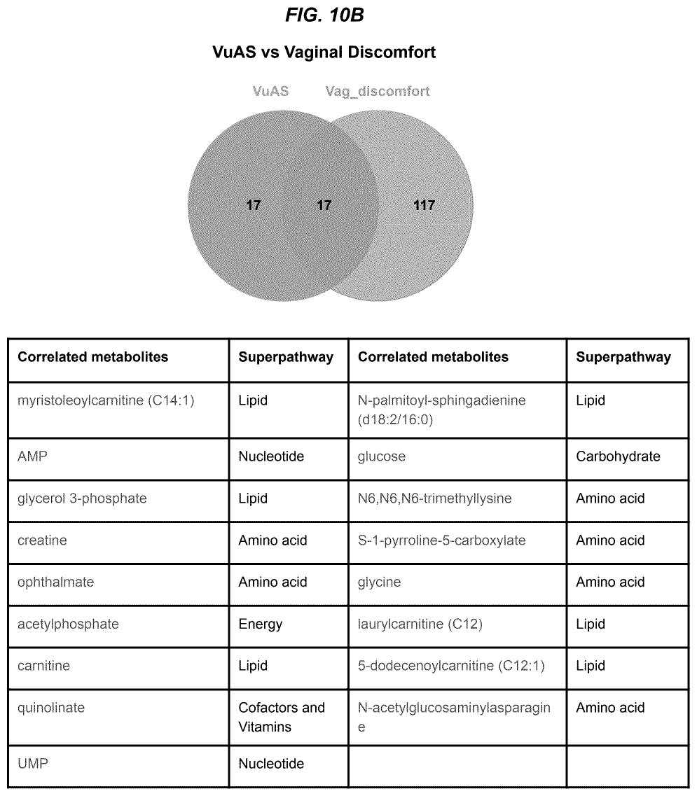

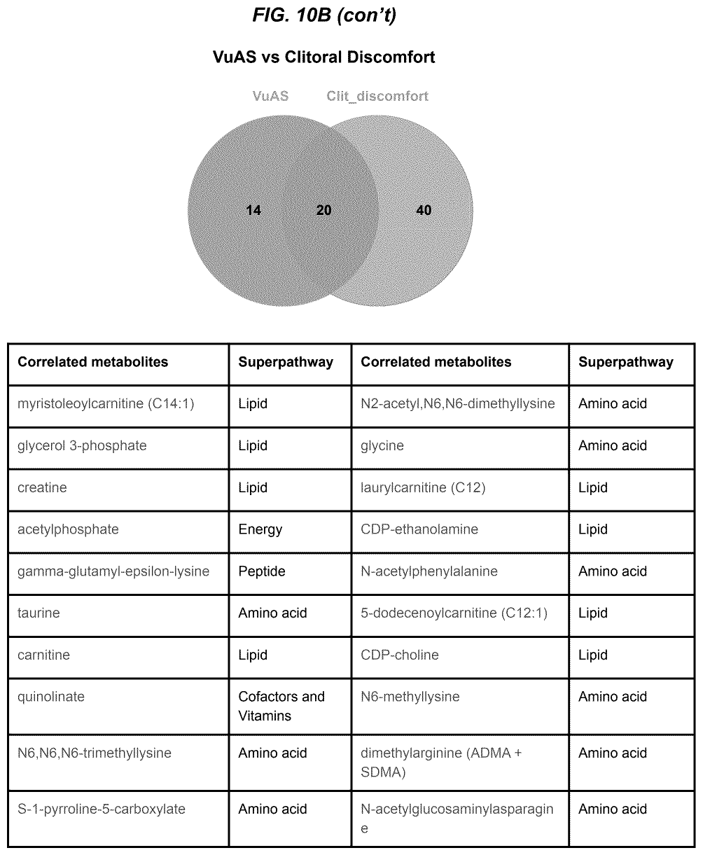

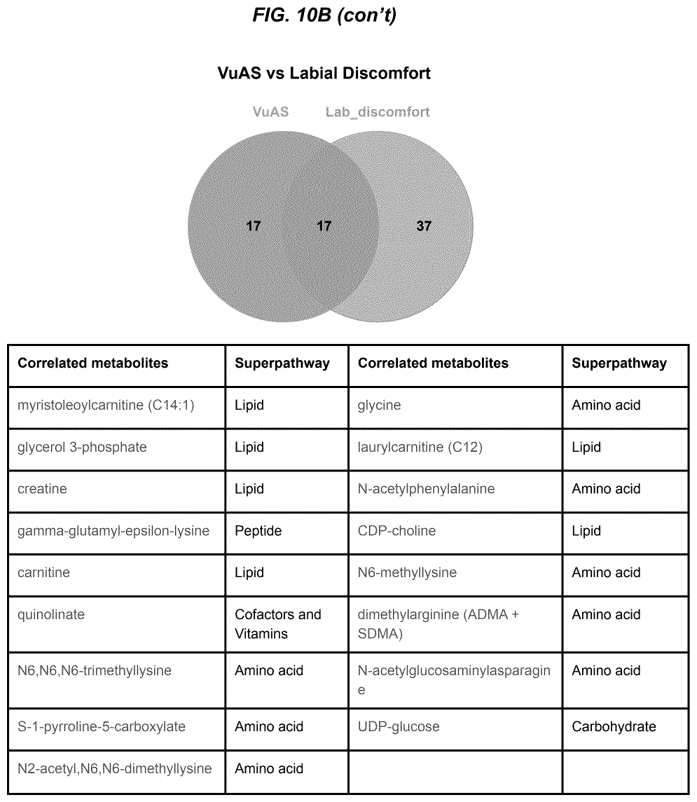

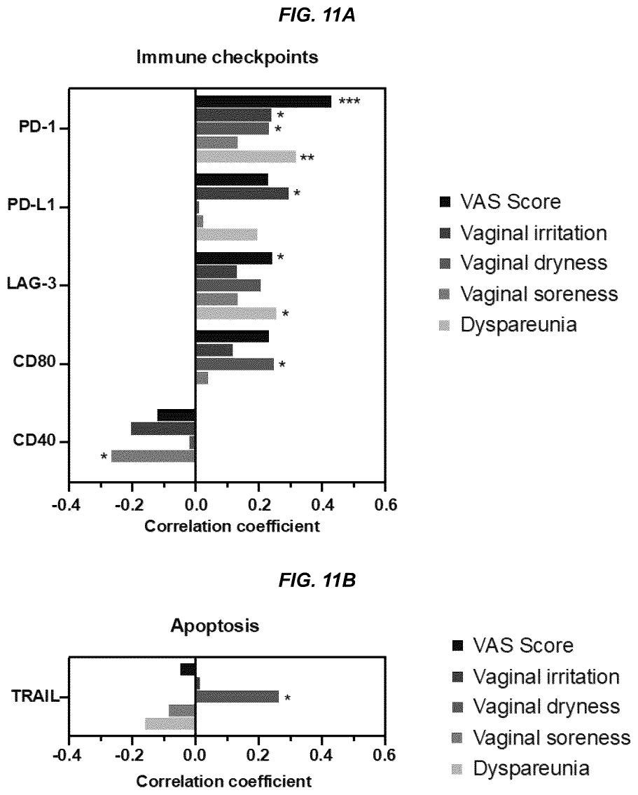

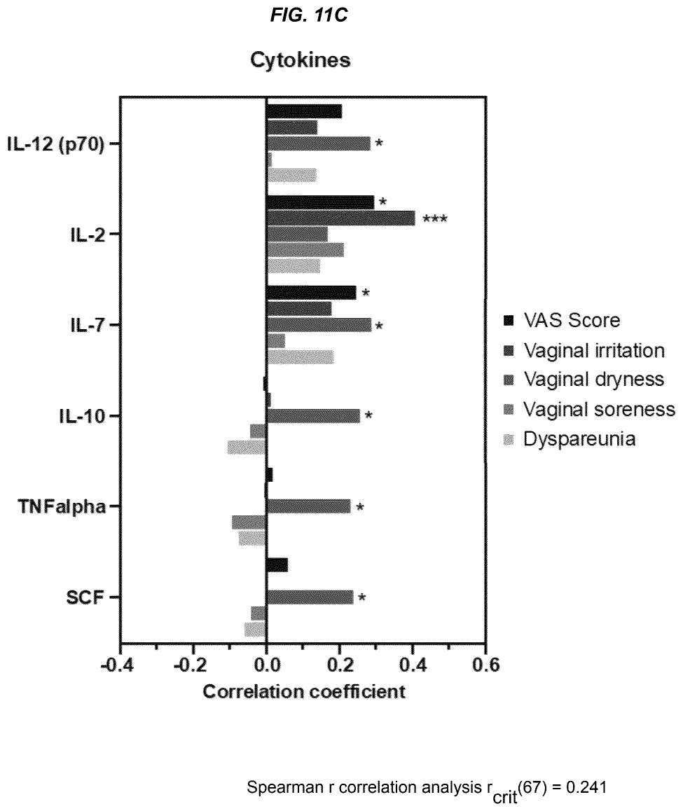

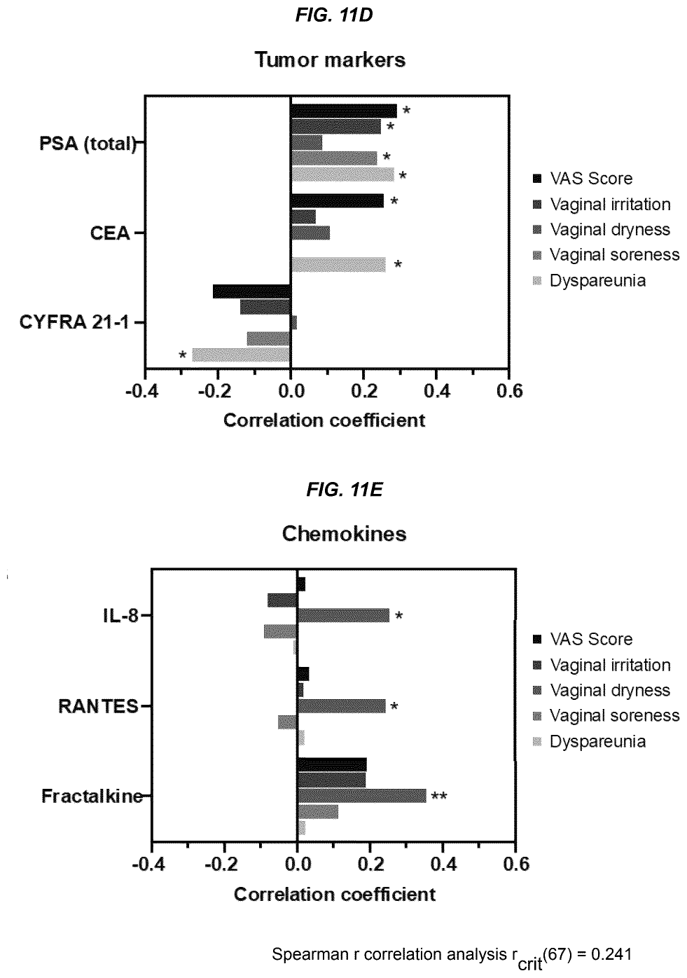

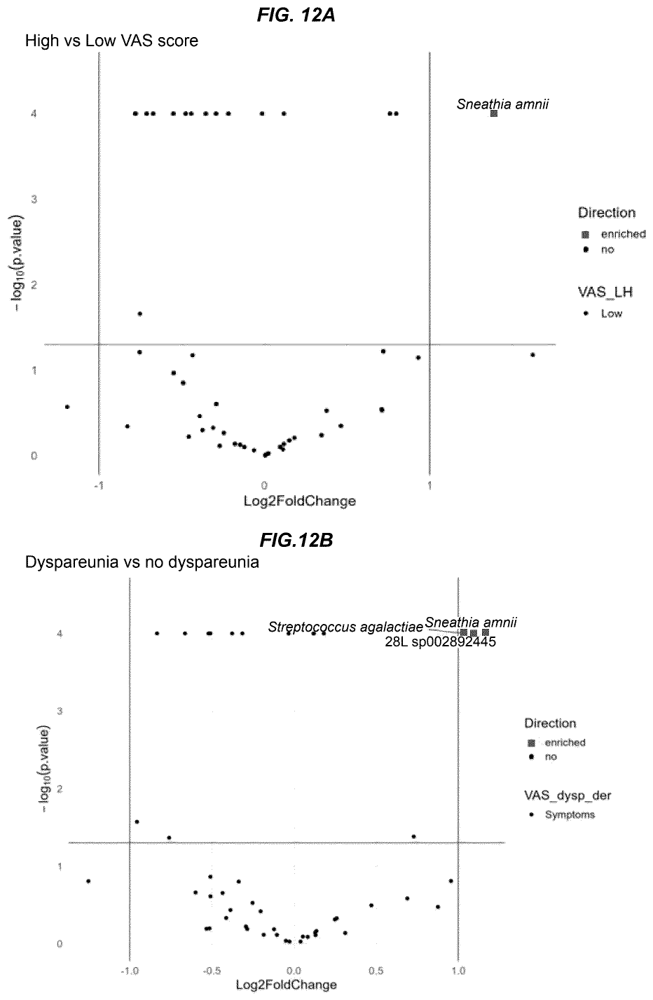

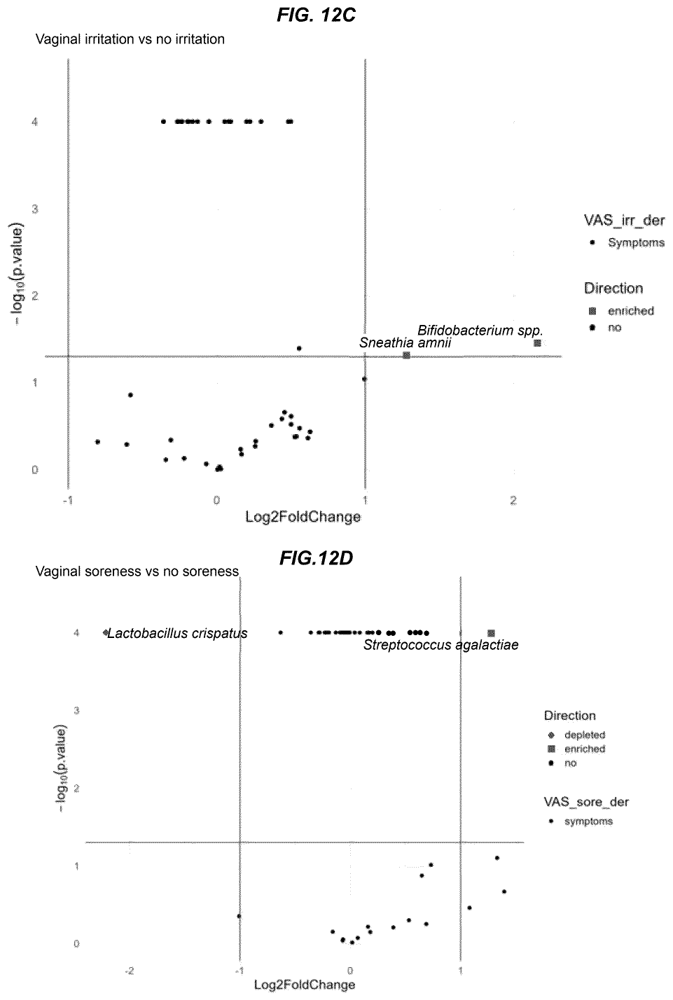

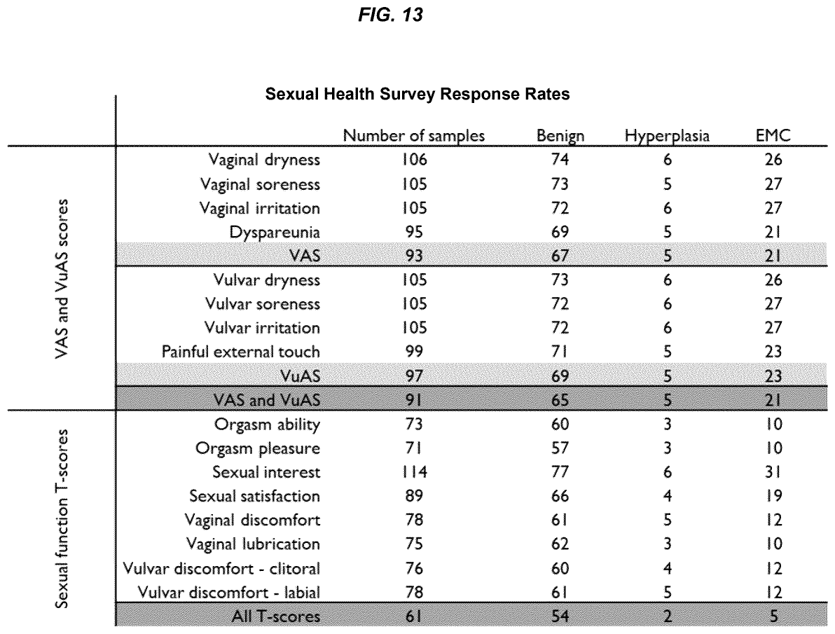

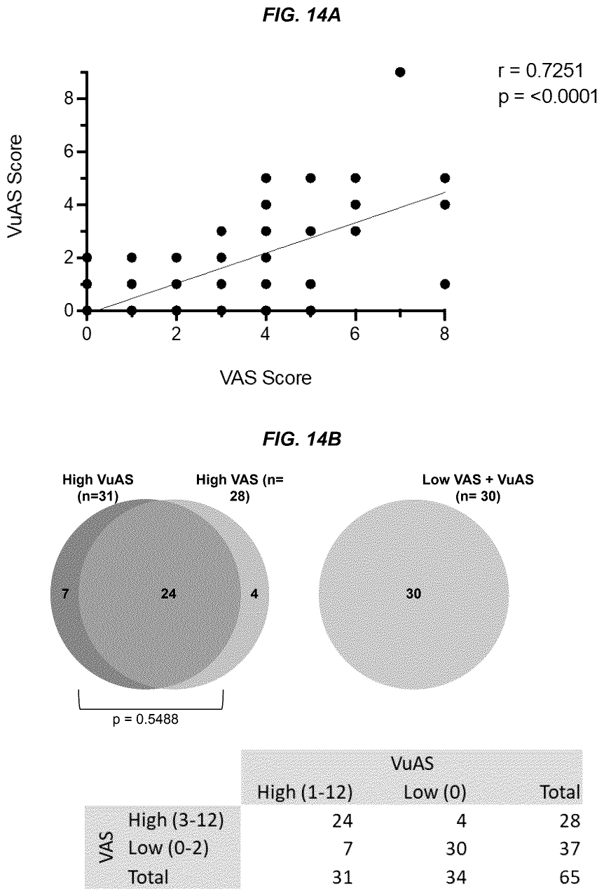

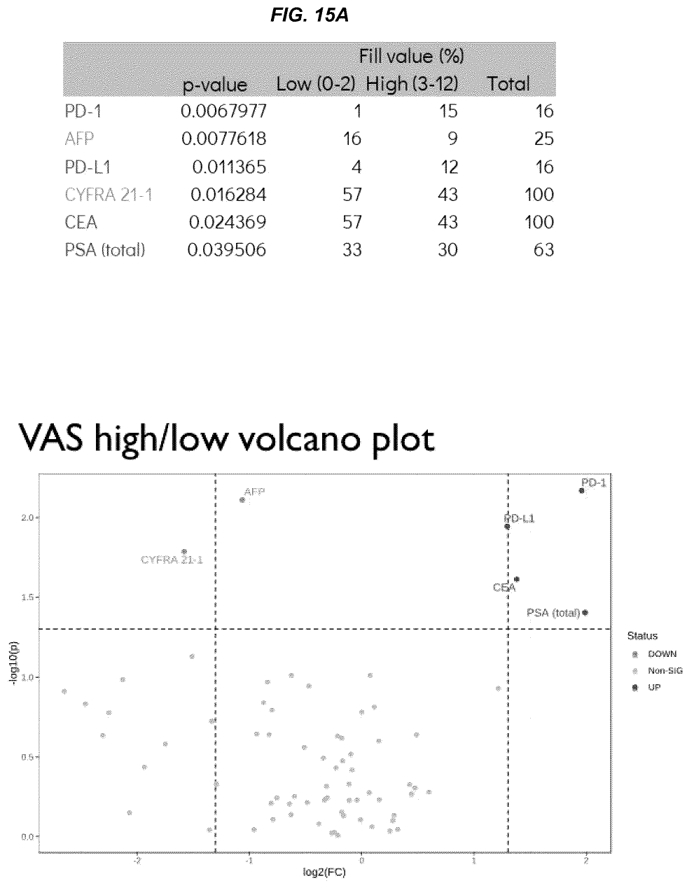

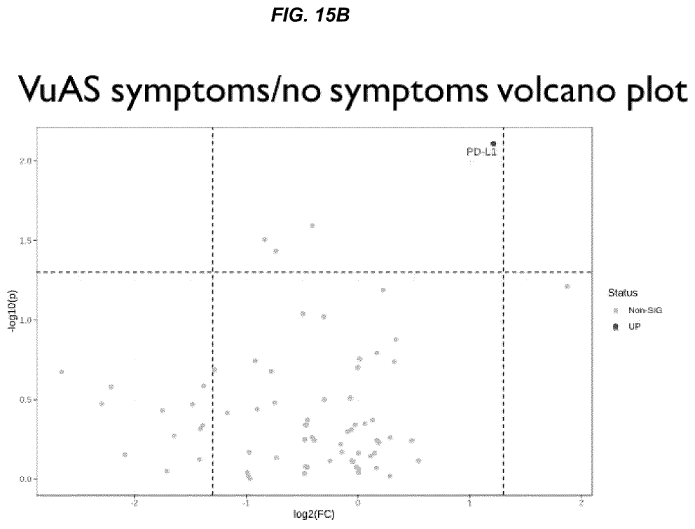

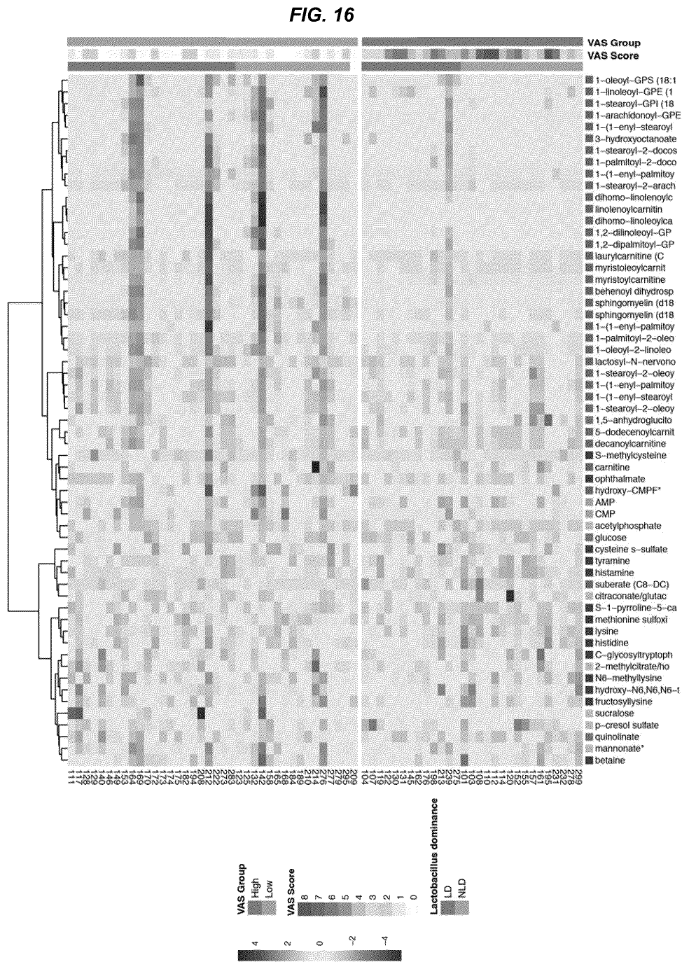

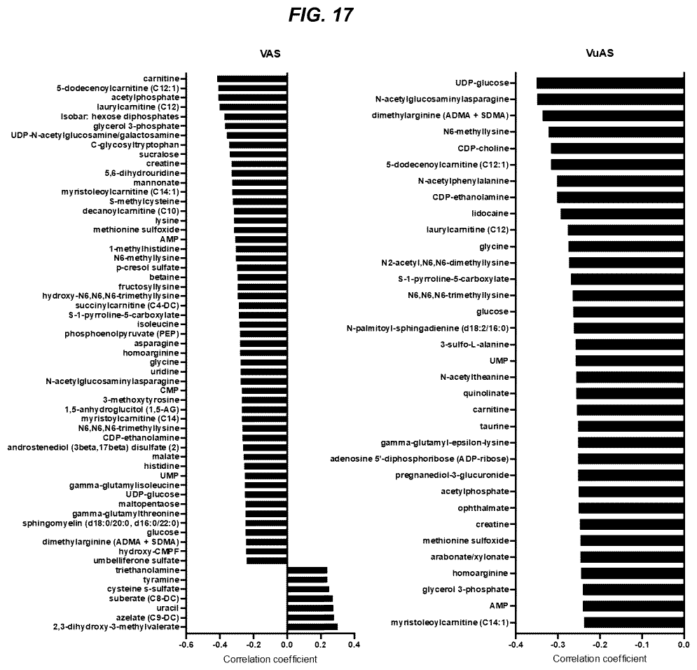

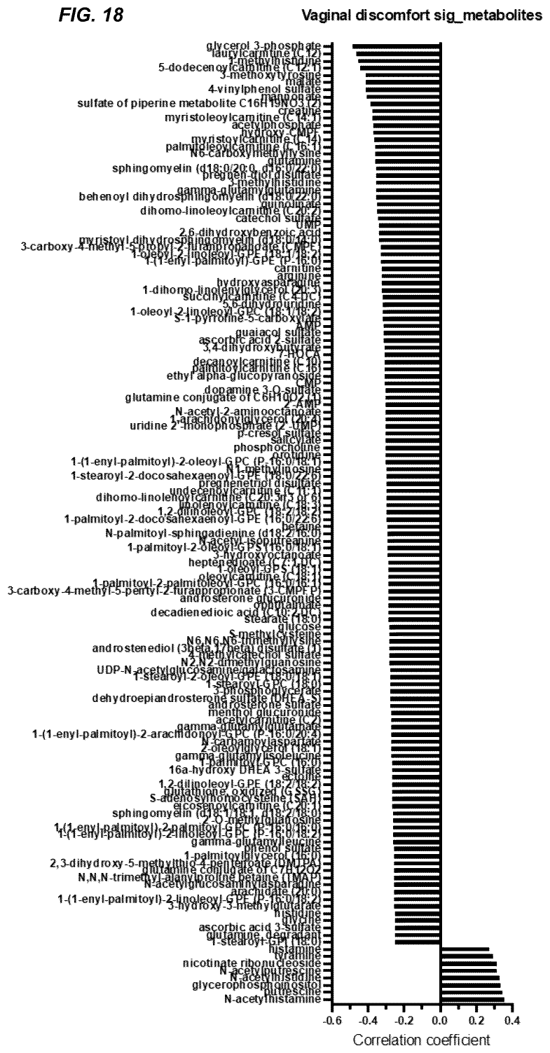

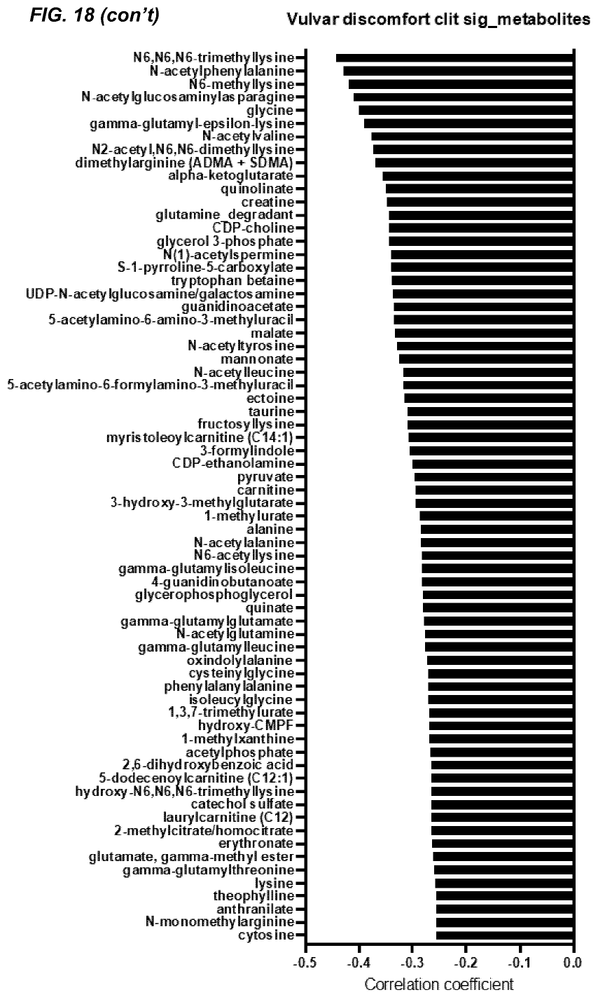

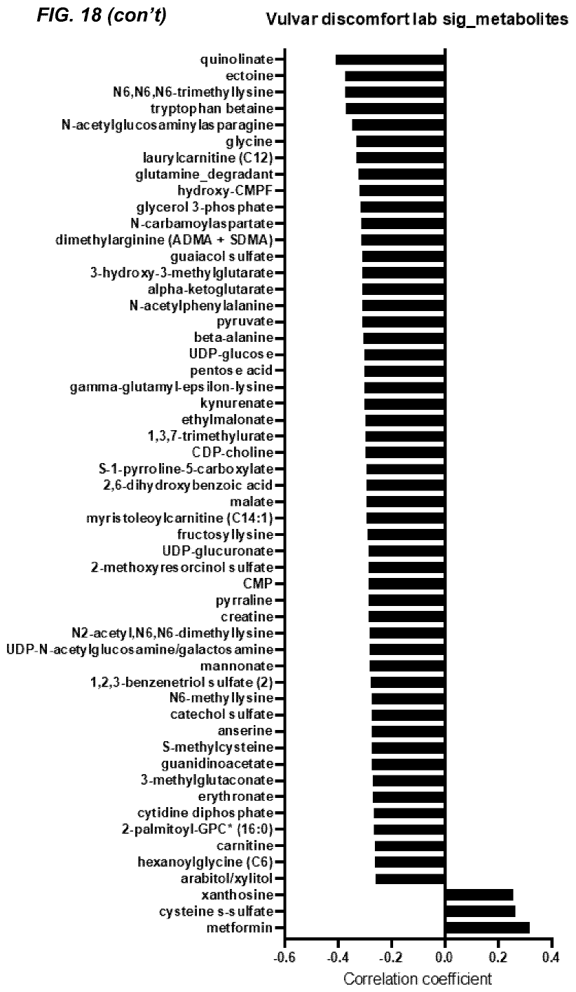

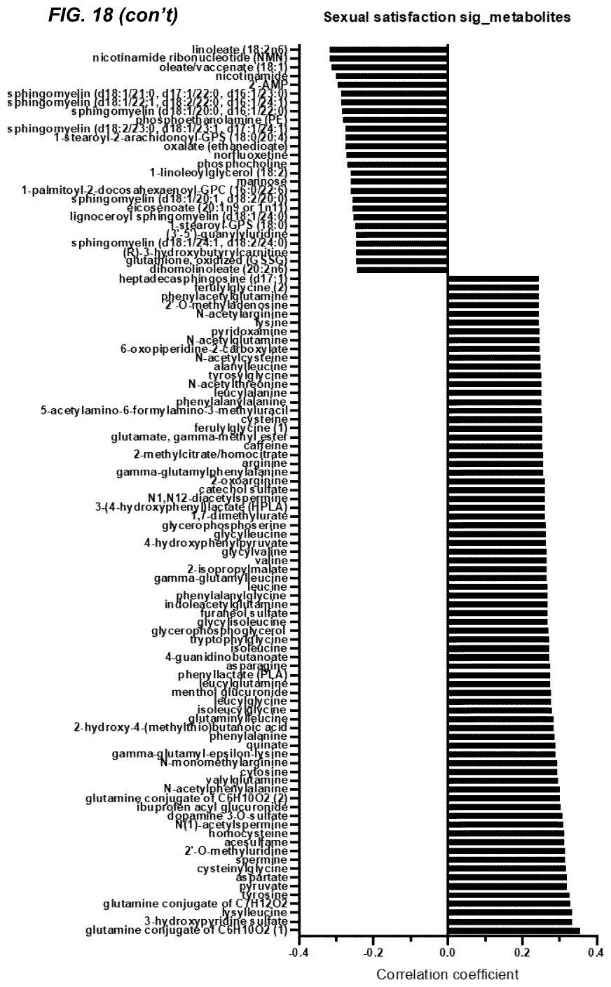

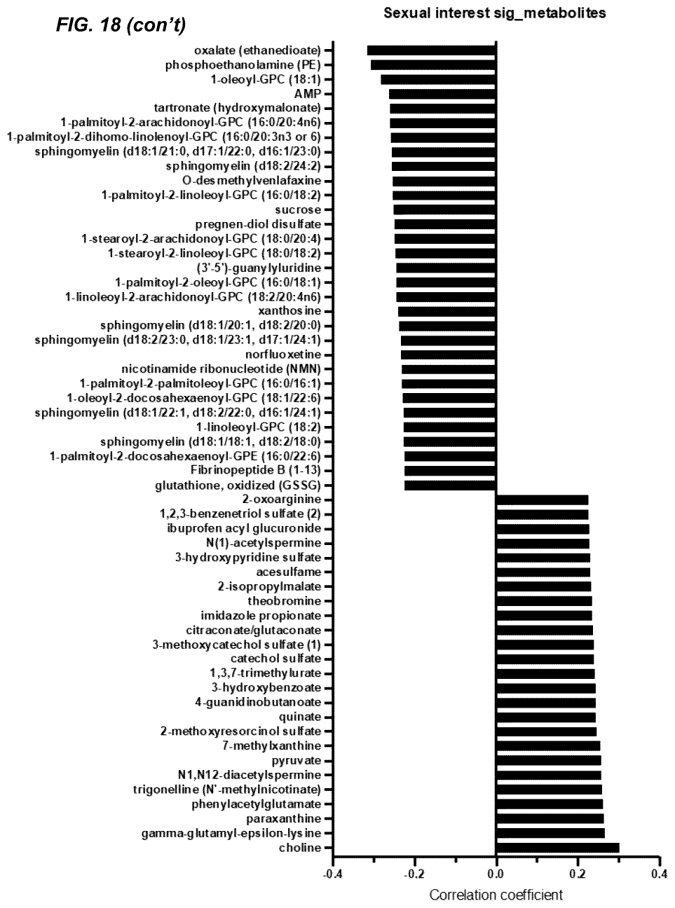

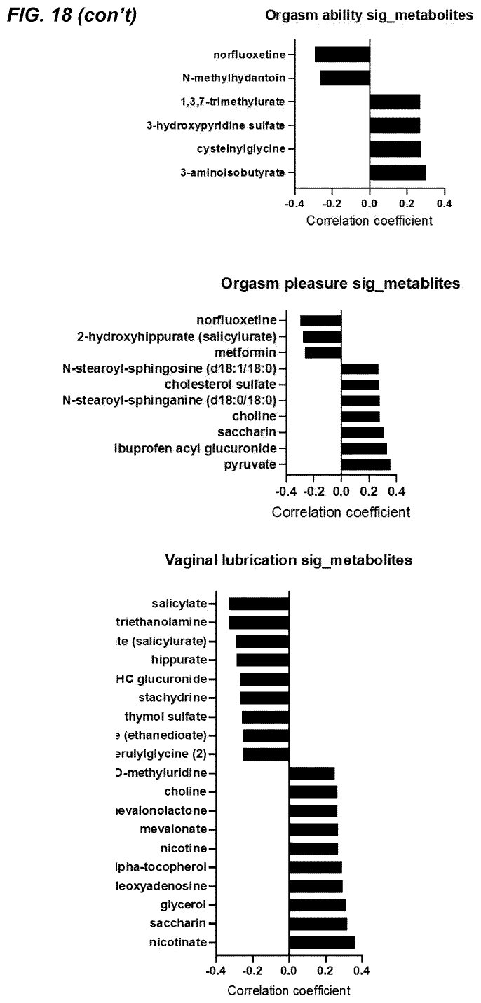

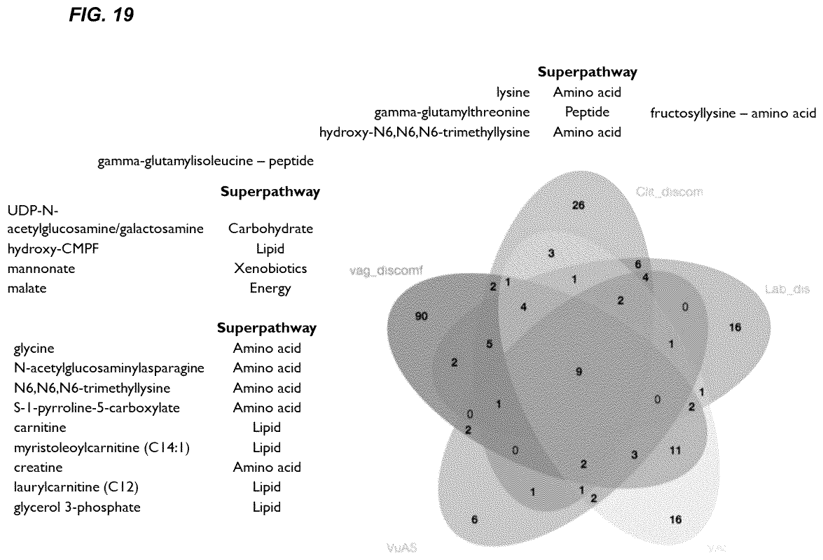

Referring now to FIGS. 1-29, the present invention features a non-invasive test for guiding treatments of vulvovaginal symptoms and female sexual dysfunction. Methods described herein may be used in pre- and postmenopausal women suffering from vulvar and vaginal symptoms, such as dryness, irritation, painful intercourse, pain during urination, etc.

The present invention features a method for providing a therapeutic solution to treat vulvovaginal symptoms in a female patient in need thereof. In some embodiments, the method comprises: determining the patient's levels of two or more biomarkers, applying the results to predetermined thresholds, wherein deviation of at least two biomarkers from the thresholds identifies at least one therapeutic solution for treating the vulvovaginal symptoms, and providing the therapeutic solution to a medical professional to guide treatment of the patient. In some embodiments, the levels of two or more biomarkers are determined by obtaining a biological sample from the patient and measuring the levels of two or more biomarkers in the biological sample obtained. In some embodiments, the biological sample comprises a cervicovaginal lavage (CVL) sample or a vaginal swab. In some embodiments, the vulvovaginal symptoms are caused by genitourinary syndrome of menopause (GSM) and may include vaginal dryness, vaginal irritation, vaginal soreness, dyspareunia, vulvar dryness, vulvar irritation, vulvar soreness, and pain. Non-limiting examples of therapeutic solutions that may be used in treatment include, but are not limited to, hormonal therapies, antimicrobials, anti-inflammatories, neuromodulators, vaginal moisturizers, antihistamines, and pelvic floor therapy.

The present invention features a method of treating vulvovaginal symptoms in a female patient in need thereof. The method comprises determining the patient's levels of two or more biomarkers and treating (e.g., estrogen-based therapies vs. vaginal lubricants or moisturizers-non-drugs) the patient based on the levels of said biomarkers. The level of two or more biomarkers may be determined by obtaining a biological sample (e.g., a cervicovaginal lavage (CVL) sample) from the patient and measuring the levels of at least two or more biomarkers in the sample obtained. In some embodiments, the two or more biomarkers are altered in the patient as compared to a healthy control.

Additionally, the present invention may feature an in vitro method of treating vulvovaginal symptoms in a female patient in need thereof. The method may comprise determining the patient's levels of two or more biomarkers and administering to the patient a treatment for the vulvovaginal symptoms if the levels of the at least two or more biomarkers deviate from a predetermined threshold. Alternatively, treatment may be administered to the patient if the levels of the at least two or more biomarkers are altered compared to a healthy control. The level of two or more biomarkers may be determined by obtaining a biological sample (e.g., a cervicovaginal lavage (CVL) sample) from the patient and measuring the levels of at least two or more biomarkers in the sample obtained.

Likewise, the present invention may feature an in vitro method of diagnosing vulvovaginal symptoms in a female patient in need thereof. The method may comprise determining the patient's levels of two or more biomarkers and diagnosing vulvovaginal symptoms in a patient if the levels of the at least two or more biomarkers deviate from a predetermined threshold. Alternatively, a diagnosis may be made if the levels of the at least two or more biomarkers are altered compared to a healthy control. The level of two or more biomarkers may be determined by obtaining a biological sample (e.g., a cervicovaginal lavage (CVL) sample) from the patient and measuring the levels of at least two or more biomarkers in the sample obtained.

In some embodiments, the aforementioned methods comprise determining the patient's level of three or more biomarkers. In some embodiments, the aforementioned methods comprise determining the patient's level of four or more biomarkers. In some embodiments, the aforementioned methods comprise determining the patient's level of five or more biomarkers. In some embodiments, the aforementioned methods comprise determining the patient's level of ten or more biomarkers. In some embodiments, the aforementioned methods comprise determining the patient's level of fifteen or more biomarkers. In some embodiments, the aforementioned methods comprise determining the patient's level of twenty or more biomarkers. In some embodiments, the aforementioned method comprises determining the patient's level of fifty or more biomarkers.

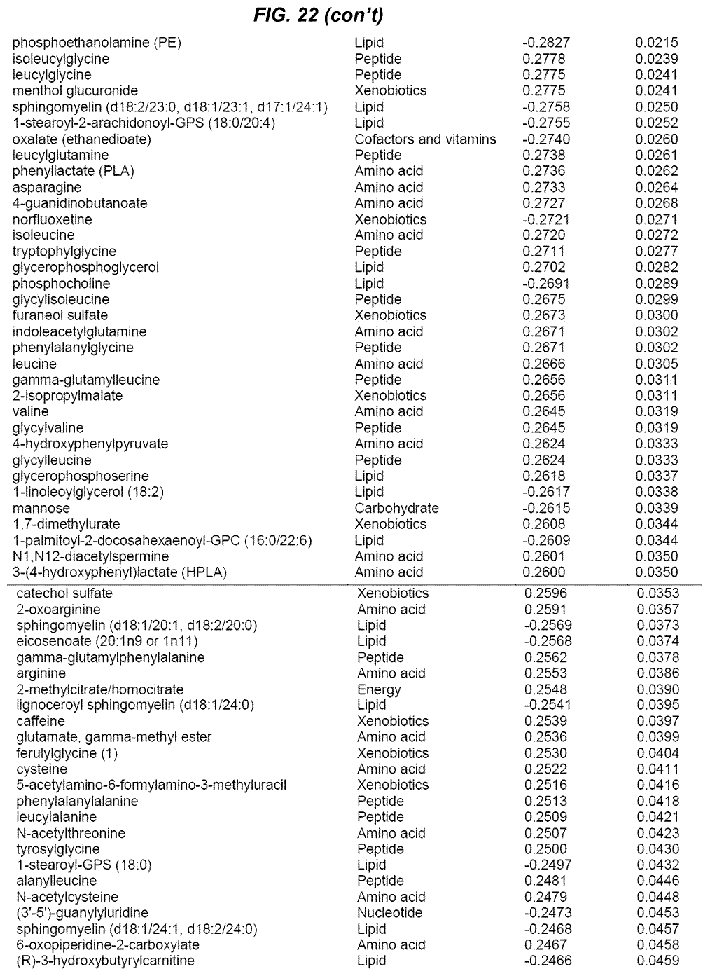

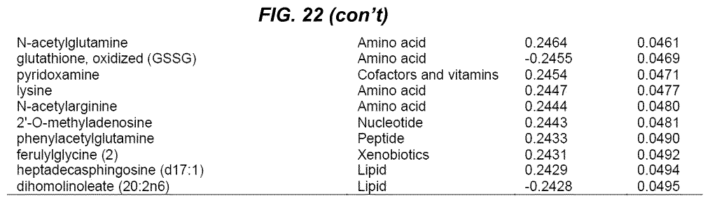

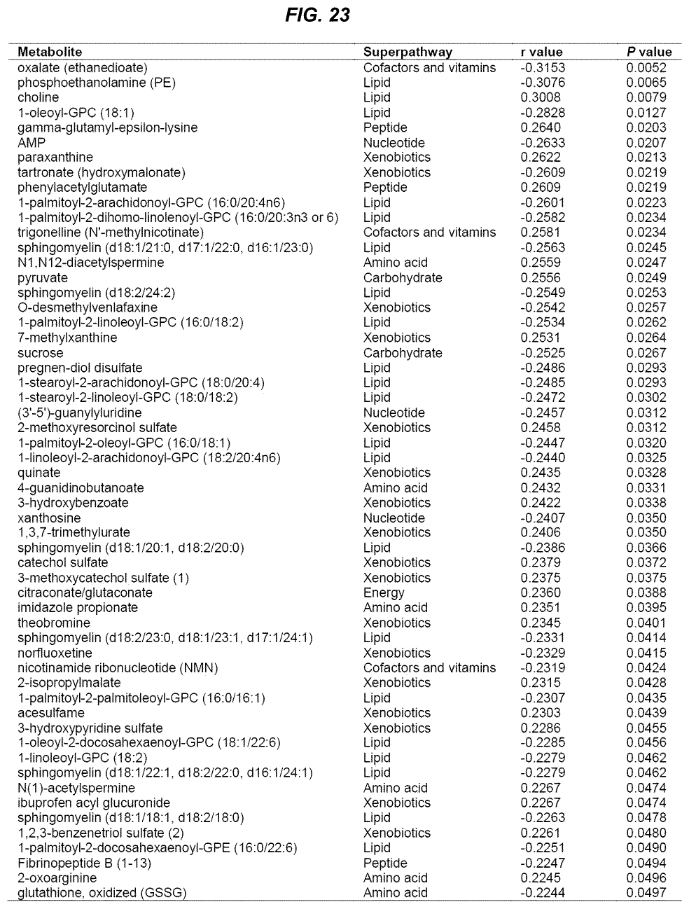

In some embodiments, biomarkers used for treating vulvovaginal symptoms in a patient may comprise two or more biomarkers selected from biomarkers listed in FIG. 22 or FIG. 23. In some embodiments, biomarkers used for treating vulvovaginal symptoms in a patient may comprise three or more biomarkers selected from biomarkers listed in FIG. 22 or FIG. 23. In some embodiments, biomarkers used for treating vulvovaginal symptoms in a patient may comprise five or more biomarkers selected from biomarkers listed in FIG. 22 or FIG. 23. In some embodiments, biomarkers used for treating vulvovaginal symptoms in a patient may comprise ten or more biomarkers selected from biomarkers listed in FIG. 22 or FIG. 23. In some embodiments, biomarkers used for treating vulvovaginal symptoms in a patient may comprise twenty or more biomarkers selected from biomarkers listed in FIG. 22 or FIG. 23.

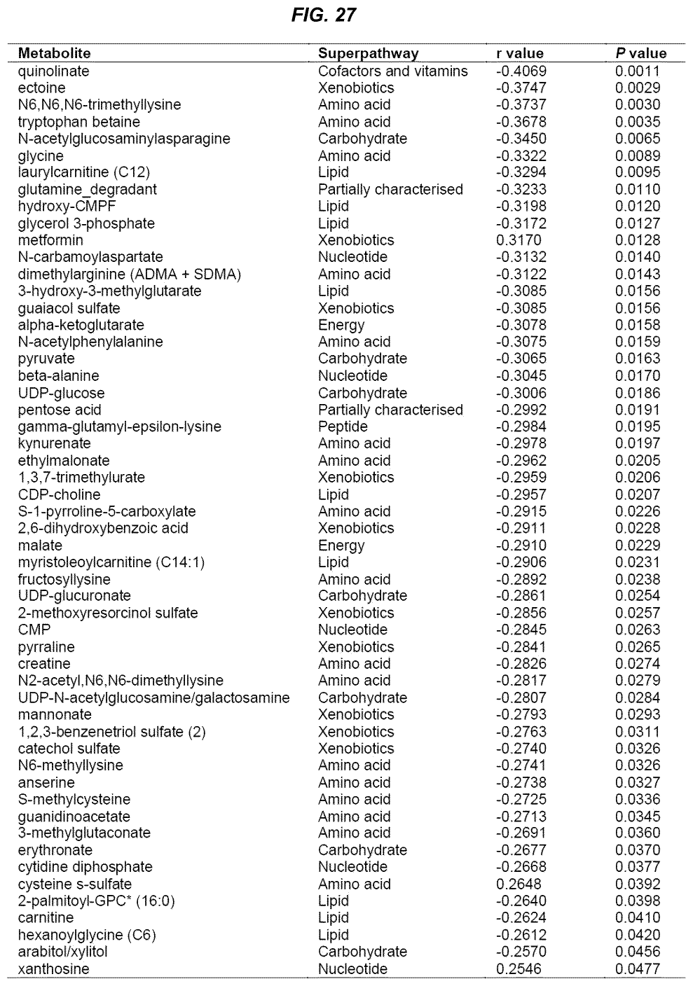

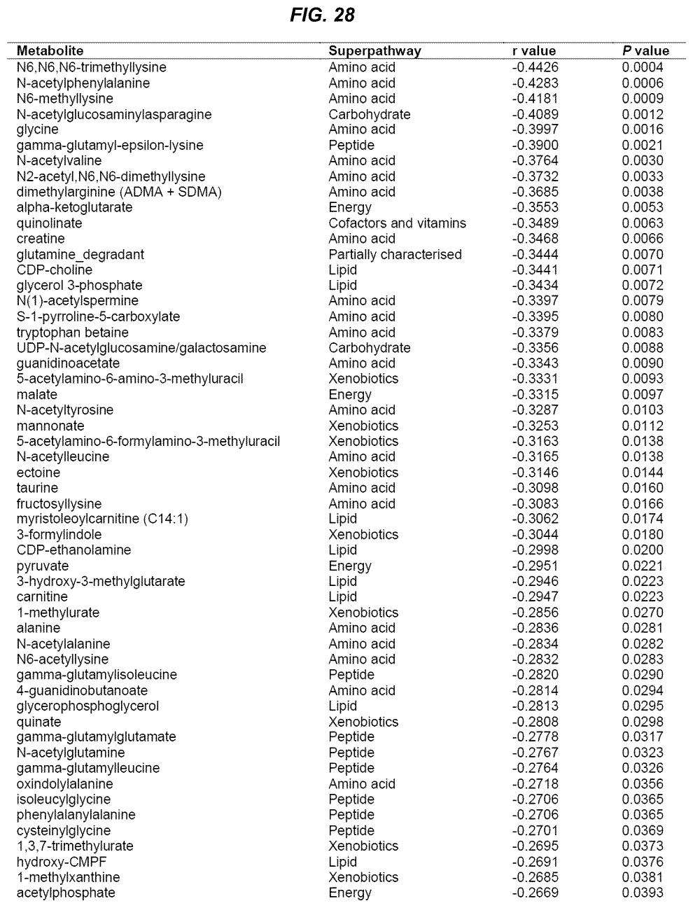

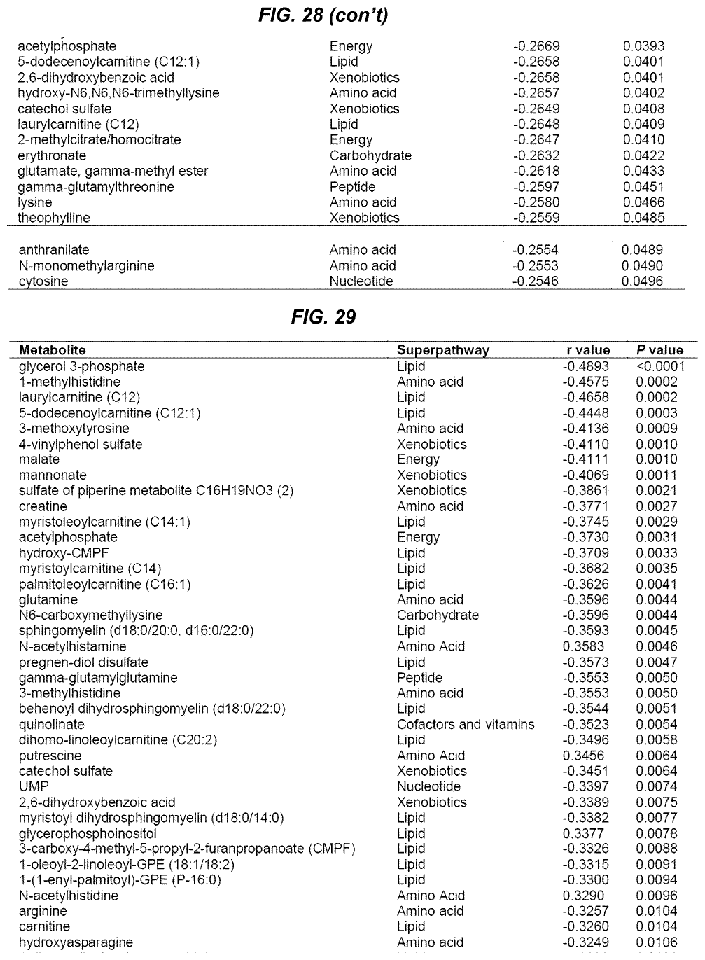

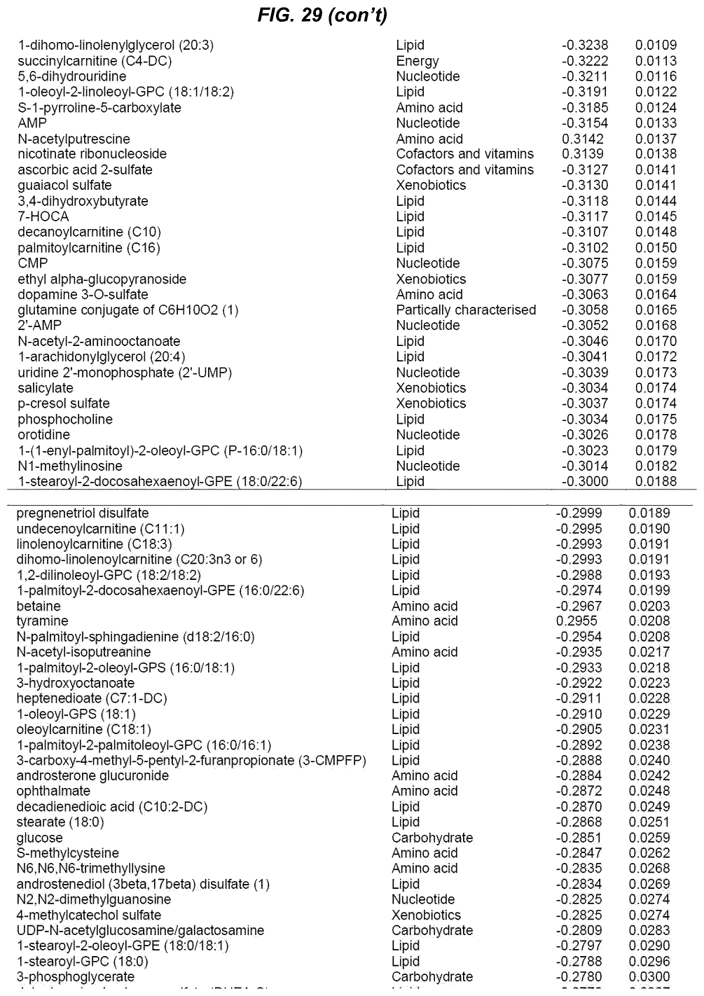

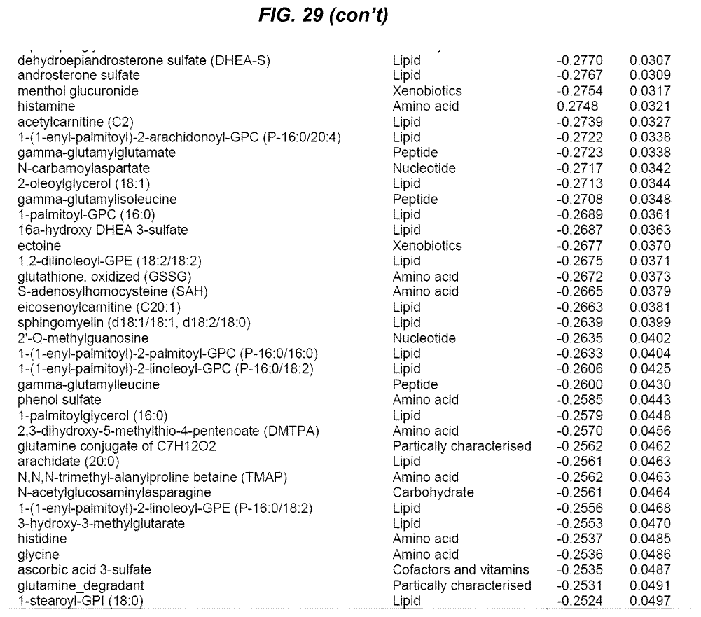

In some embodiments, a downregulation of biomarkers associated with lipids may indicate an increase in vaginal dryness, vaginal soreness, vaginal irritation, dyspareunia, or a combination thereof. Biomarkers that may indicate vaginal discomfort may be selected from two or more biomarkers listed in FIG. 29. In other embodiments, a downregulation of biomarkers associated with amino acids may indicate an increase in vulvar dryness, vulvar soreness, vulvar irritation, painful external touch, or a combination thereof. Biomarkers that may indicate vulvar discomfort may be selected from two or more biomarkers listed in FIG. 27 (vulvar discomfort-labial) or FIG. 28 (vulvar discomfort-clitora).

In certain embodiments, the present invention may feature a method of treating vaginal dryness in a female patient in need thereof. The method may comprise determining the patient's levels of two or more biomarkers (e.g., CA15-3, CD80, LAG-3, Fracktiline, IL-12, SCF as well as biomarkers listed in FIG. 22 and FIG. 23) and administering a treatment (e.g., vaginal lubricant or moisturizer) to the patient based on the levels of said biomarkers. The level of two or more biomarkers may be determined by obtaining a biological sample (e.g., a cervicovaginal lavage (CVL) sample) from the patient and measuring the levels of at least two or more biomarkers in the sample obtained.

In some embodiments, the vulvovaginal symptoms are caused by genitourinary syndrome of menopause (GSM) or past cancer-related treatments (e.g., radiation, chemotherapy, etc.). In some embodiments, the vulvovaginal symptoms are caused by benign gynecologic conditions. Non-limiting examples of benign gynecologic conditions include but are not limited to endometriosis, adenomyosis, and fibroids. In some embodiments, the vulvovaginal symptoms comprise vaginal dryness, vaginal irritation, vaginal soreness, dyspareunia, vulvar dryness, vulvar irritation, vulvar soreness, and pain.

In some embodiments, the biological sample may comprise cervicovaginal lavage (CVL) sample, urine, blood, vaginal swab, or cervical secretions (e.g., menstrual fluid; e.g., collected using a menstrual cup or similar devices). In some embodiments, two biological samples may be obtained from the subject, e.g, a CVL sample and a blood sample.

The biomarkers may comprise immune mediators, metabolites (e.g., xenobiotics), bacterial abundance, or a combination thereof. In some embodiments, the biomarkers comprise a combination of immune mediators and metabolites. In some embodiments, the biomarkers comprise a combination of immune mediators and bacterial abundance. In some embodiments, the biomarkers comprise a combination of metabolites and bacterial abundance. In some embodiments, the biomarkers comprises a combination of immune mediators (e.g., immune protein), metabolites (e.g., lipids), and bacterial abundance.

For example, in some embodiments, the biomarkers may include metabolite biomarkers (e.g., amino acid metabolite biomarkers; including the biogenic amines putrescine and N-acetylputrescine, and three histidine derivatives, histamine, N-acetylhistamine, and imidazole propionate) and bacterial abundance biomarkers (e.g., M. lornae, Prevotella amnii, Fannyhessea vaginae, Sneathia sanguinegens, Prevotella sp., Bifidobacterium sp., and UBA1822 sp900545365 (Dialisteraceae family)). In some embodiments, the aforementioned biomarkers may be associated with vaginal discomfort.

In some embodiments, the immune mediators (e.g., immune proteins) comprise immune proteins involved in T-cell activation and regulation of inflammation. Non-limiting examples of immune proteins, including proteins involved in the regulation of inflammation, include but are not limited to . . . programmed cell death protein 1 (PD-1), programmed death-ligand 1 (PD-L1), interleukin-2 (IL-2), IL-7, IL-12, IL-10, IL-8, tumor necrosis factor-alpha (TNF-alpha), stem cell factor (SCF), lymphocyte activation gene 3 (LAG-3), CD80, CD40, TNF-related apoptosis-inducing ligand (TRAIL), prostate-specific antigen (PSA), alpha-fetoprotein (AFP), cytokeratin fraction 21-1 (CYFRA 21-1), and carcinoembryonic antigen (CEA).

In some embodiments, the metabolites comprise amino acids, nucleotides, lipids (such as glycerophospholipids, fatty acids, or sphingolipids), xenobiotics, or a combination thereof. In some embodiments, the metabolites comprise one or more compounds selected from different metabolite classes. For example, suitable metabolites may include carnitines and related compounds such as laurylcarnitine (C12), 5-dodecenoylcarnitine, carnitine, myristoleoylcarnitine (C14:1), and decanoylcarnitine (C10); organic acids and derivatives such as mannonate, tricarballyate, and citraconate/glutaconate; amino acids and amino acid derivatives such as N-acetylglucosaminylasparagine, N-acetylglycine, N-acetylaspartate (NAA), N-acetylphenylalanine, N-delta-acetylornithine, N5-methyllysine, hydroxy-N6,N6,N6-trimethyllysine, N-acetylalanine, and gamma-glutamyl-epsilon-lysine; nucleotides and nucleotide derivatives such as adenosine monophosphate (AMP) and 2′O-methylguanosine; xenobiotics such as p-cresol sulfate; and biogenic amines such as histamine. In further embodiments, the metabolites may include lipids such as behenoyl dihydrosphingomyelin (d18:0/22:0), sphingomyelin (d18:0/20:0, d16:0/22:0), 1-stearoyl-GPS (18:0), 1-palmitoyl-2-docosahexaenoyl-GPE (16:0/22:6), sphingomyelin (d18:1/22:1, d18:2/22:0, d16:1/24:1), sphingomyelin (d18:2/23:0, d18:1/23:1, d17:1/24:1), and sphingomyelin (d18:1/20:1, d18:2/20:0, d18:1/21:0, d17:1/22:0, d16:1/23:0).

In some embodiments, the metabolites comprise amino acids, nucleotides, glycerophospholipids, fatty acids, and sphingolipids. In some embodiments, the metabolites comprise xenobiotics. In certain embodiments, the metabolites comprise N6-methyllysine, hydroxy-N6,N6,N6-trimethyllysine, sucralose, laurylcarnitine (C12), or a combination thereof.

In some embodiments, the bacterial abundance of Sneathia amnii/vaginalis, Megasphaera lornae (28 L sp002892445), Group B Streptococcus (Streptococcus agalactiae), Lactobacillus crispatus, or other bacterial vaginosis (BV)-associated bacteria (Bifidobacterium/Gardnerella spp., Prevotella spp., Fannyhessea vaginae, Peptoniphilus spp., Porphyromonas spp.) is measured.

In some embodiments, Lactobacillus crispatus may be reduced in female patients with vulvovaginal symptoms (e.g., vaginal soreness) as compared to a healthy female patient. In other embodiments, Sneathia amnii, Megasphaera lornae, Streptococcus agalactiae, Bifidobacterium sp., or a combination thereof may be increased in female patients with vulvovaginal symptoms (e.g., soreness, irritation, dyspareunia,) as compared to a healthy female patient.

In some embodiments, treatments may include but are not limited to hormonal therapies, antimicrobials, anti-inflammatoires, neuromodulators, vaginal moisturizers, antihistamines, or pelvic floor therapy. In some embodiments, treatments may include hormone therapies such as estrogen, estrogen receptor, or androgen therapies. For example, menopause hormone therapy may be used during perimenopause in premenopausal women. The treatments described herein for use in accordance with the present invention may comprise different formulations (e.g., local delivery through creams vs systemic delivery). Non-drug treatments would be the use of moisturizers, creams, etc.

In certain embodiments, at least one of the two or more biomarkers comprises histamine. In other embodiments, the two or more biomarkers comprise histamine and at least one carnitine or a derivative thereof, wherein the carnitine derivative is selected from the group consisting of laurylcarnitine (C12), 5-dodecenoylcarnitine, myristoleoylcarnitine (C14:1), and decanoylcarnitine (C10). In some embodiments, when histamine is increased relative to a predetermined threshold and the at least one carnitine or derivative thereof is decreased relative to a predetermined threshold, the therapeutic solution comprises an antihistamine. In other embodiments, when histamine is not elevated relative to a predetermined threshold and the levels of carnitine or derivatives thereof are not decreased, the biomarker profile is more consistent with genitourinary syndrome of menopause (GSM). In such embodiments, the therapeutic solution comprises an estrogen therapy, a selective estrogen receptor modulator (SERM), or dehydroepiandrosterone (DHEA).

In certain embodiments, the two or more biomarkers comprise two or more immune proteins as described above. In some embodiments, when levels of two or more of these immune proteins are increased relative to a predetermined threshold, this is indicative of vaginal dryness or vaginal irritation, and the therapeutic solution comprises one or more of lubricants, moisturizers, or hormonal therapy, administered locally or systemically. In cases of vaginal irritation, further investigation may be required to identify the source of irritation, which may guide additional or combination therapies.

### Example 1

The following is a non-limiting example of the present invention. It is to be understood that said example is not intended to limit the present invention in any way. Equivalents or substitutes are within the scope of the present invention.

Study participants: Seventy-nine participants undergoing hysterectomy for benign gynaecological conditions were recruited. Briefly, inclusion criteria included: female of any race or ethnicity 18 years or older; exclusion criteria included: currently menstruating; currently on antibiotics, antifungals, antivirals or topical steroids or within the previous 12 weeks; current vaginal infection (including bacterial vaginosis and candidiasis), or vulvar, urinary tract, or sexually transmitted infections or within the previous 3 weeks; history of or current self-reported diagnosis of genital herpes; inflammatory bowel conditions such as Crohn's, inflammatory bowel disease, diverticulitis, and C. difficile infection; use of douching substances, vaginal medications or suppositories, feminine deodorant sprays, wipes, or lubricants within the past 48 hours; sexual intercourse within the past 48 hours; hepatitis; and being HIV-positive. Women who did not complete any sexual health surveys were also excluded. Physician's pelvic exams, medical records, and/or self-reported surveys were used to verify exclusion criteria and collect demographic, socioeconomic, and medical history data. The term “women” used throughout the study refers to the biological sex of participants, not their gender identity.

Sexual health surveys: Participants completed three self-reported surveys to assess their vulvovaginal symptoms and sexual health: Vaginal Assessment Scale (VAS), Vulvar Assessment Scale (VuAS), and Patient-Reported Outcome Measurement System Sexual Function and Satisfaction v2.0 Brief Profile (PROMIS SexFS).

The VAS and VuAS are each four-item measurements that quantify and rate (none, mild, moderate, or severe) the patient's perception of dryness, soreness, irritation without sexual activity, and pain with sexual activity (dyspareunia or painfulness to touch with external stimulation) for the vaginal and vulvar areas. Each symptom is scored by severity from 0 (none) to 3 (severe) and summed to calculate total VAS and VuAS scores (range 0-12) (see Table 1A). Total scores were calculated for participants who answered all individual questions on the VAS and VuAS surveys. For some analyses, participants were dichotomized based on the median total score for each survey. This resulted in groups of low VAS (0-2) and high VAS (3-12), and low VuAS(0) and high VuAS (1-12). Higher total scores indicate high severity of one symptom or low severity of many symptoms and overall worse sexual function than low total scores.

Table 1A shows VAS and VuAS Scores. Participants were asked to rate each symptom as none (0), mild (1), moderate (2), or severe (3).

| VAS Scores | | | VuAS Scores | | |
|---|---|---|---|---|---|
| Symptoms | Score | n | Symptoms | Score | n |
| Vaginal dryness | 0-3 | 74 | Vulvar dryness | 0-3 | 73 |
| Vaginal Soreness | 0-3 | 73 | Vulvar Soreness | 0-3 | 72 |
| Vaginal irritation | 0-3 | 72 | Vulvar irritation | 0-3 | 72 |
| Dyspareunia | 0-3 | 69 | Painful external touch | 0-3 | 71 |
| VAS score | 0-12 | 67 | VuAS score | 0-12 | 69 |

The PROMIS SexFS survey assesses 14 items in eight domains: sexual interest, sexual satisfaction, orgasm ability, orgasm pleasure, vaginal lubrication, vaginal discomfort, vulvar discomfort clitoral, and vulvar discomfort labial. Final scores were expressed as T-scores (See Table 1B), which were calibrated with item response theory. Patients were not dichotomized based on the PROMIS SexFS scores.

Table 1B shows T-scores.

| Variables | n |
|---|---|
| Sexual interest | 77 |
| Sexual satisfaction | 66 |
| Orgasm ability | 60 |
| Orgasm pleasure | 57 |
| Vaginal lubrication | 62 |
| Vaginal discomfort | 61 |
| Vulvar discomfort (clitoral) | 60 |
| Vulvar discomfort (labial) | 61 |
| All t-scores | 54 |

Sample collection and processing: Vaginal swabs and cervicovaginal lavages (CVLs) were collected by a surgeon in the operating room during the standard-of-care hysterectomy procedure. CVLs were collected and processed. Briefly, CVLs were collected using 10 ml of sterile 0.9% saline solution (Teknova, Hollister, CA), immediately placed on ice, and frozen at −80° C. within 1 hour. Prior to downstream analyses, CVLs were thawed on ice and cleared by centrifugation (700×g for 10 min at 4° C.). Vaginal swabs were collected using an eSwab collection system with Amies transport medium (COPAN Diagnostics, Murrieta, CA) and DNA was extracted from swabs using DNeasy PowerSoil kit (Qiagen, Germantown, MD) following the manufacturer's protocol. CVLs and extracted DNA were aliquoted to avoid multiple freeze-thaw cycles and stored at −80° C.

Quantification of soluble metabolites and proteins: CVL samples were utilized for metabolome and immunoproteome analysis. Briefly, soluble metabolites in CVL samples were identified and quantified by Metabolon, Inc. (Durham, NC) using a Waters ACQUITY ultra-performance liquid chromatography (UPLC) and a Thermo Scientific Q-exactive high resolution/accurate mass spectrometer interfaced with a heated electrospray ionization (HESI-II) source and Orbitrap mass analyzer operated at 35,000 mass resolution. Metabolon's Laboratory Information Management Systems (LIMS) was used for compound identification.

Levels of 71 proteins were measured in CVL samples using the Milliplex MAP Magnetic Bead Immunoassays: Human Cytokine Chemokine Panel 1, Human Circulating Cancer Biomarker Panel 1, and Human Immuno-Oncology Checkpoint Protein Panel 1 (Millipore, Billerica, MA) in accordance with the manufacturer's protocols. Data were collected using a Bio-Plex 200 instrument and analyzed using Manager 5.0 software (Bio-Rad, Hercules, CA). IL-36y (IL-1F9) levels were measured by enzyme-linked immunosorbent assay using a Human IL-36y ELISA kit (RayBiotech, Norcross, GA) in accordance with the manufacturer's protocol. Concentration values below the detection limit were substituted with 0.5 of the minimum detectable concentration provided in the manufacturer's protocol. Metabolite and protein concentrations were log₁₀ transformed and auto-scaled (mean-centred and divided by the standard deviation) prior to bioinformatics analysis.

16S rRNA sequencing analysis and taxonomic classification: Extracted DNA from clinician-collected vaginal swabs underwent 16S rRNA gene sequencing. Bacterial 16S rRNA gene was amplified using Primers for the V4 region, Golay barcode-tagged forward primer, and Earth Microbiome Project (EMP) primers (515F-806R) for reverse primers and sequenced on the Illumina MiSeq benchtop sequencing platform (Illumina, San Diego, CA).

Microbial DNA sequencing data were processed and analyzed using the QIIME 2 version 2022.2 and demultiplexed using the q2-demux plugin. Human reads were filtered out, retaining 13,145,634 sequences. QIIME 2 diversity plugin was used to confirm that sampling depth, set to 3,714, was representative of the microbiome's diversity and to compare Faith's phylogenetic diversity (PD) and evenness. DADA2 was run through the q2-dada2 plugin, where sequence data were quality-filtered, trimmed to forward 240 and reverse 220 nt, merged, and denoised to generate amplicon sequence variants (ASVs). Taxonomy was assigned to reads using the q2-feature-classifier plugin with the classify-sklearn naive Bayes classifier against the Genome Taxonomy Database (GTDB), version r202. Taxonomic class weights utilized the pluginq2-clawback. Sequences that failed to classify at the phylum level were discarded, and ASVs with fewer than 4 reads and 10% prevalence were filtered prior to downstream analysis. Centred log ratio (CLR) was not applied prior to classification or diversity analyses. Rarefaction was applied to avoid introducing library size bias and resulted in the exclusion of 4 vaginal samples out of the 79 total samples.

Integrating global metabolic profiles with VAS and VuAS surveys: Principal component analysis (PCA) of metabolomics data was performed using Prism 9.0 (GraphPad, San Diego, CA). This analysis reduces the dimensionality in the data by creating a small number of principal components (PC) that capture most of the variance in the data. Statistical differences between the high and low VAS or VuAS groups for individual components were assessed using Welch's t-test. Hierarchical clustering analysis was performed using MetaboAnalyst 5.0 to depict the relationship between levels of the top 50 significant metabolites, determined by t-test, and VAS and VuAS patient groups. Clustering was based on Euclidean distance and Ward linkage for metabolites and supervised clustering for patient samples by VAS group.

Volcano plots and metabolite enrichment analysis: Differences in metabolite and protein levels between patients in high or low VAS and VuAS groups were determined using fold change (FC) analysis and two-sample t-tests. Unequal group variance, a fold change threshold of 2, and a P value threshold of 0.05 was used for the analysis. Results were combined and graphically presented as volcano plots. Enrichment analysis compared metabolite levels between the low and high VAS groups to The Small Molecule Pathway Database, containing metabolic pathways only found in humans. The enrichment ratio was calculated by dividing the observed number of metabolite hits by the expected number of hits within a particular metabolic pathway. Significance was determined as P<0.05. Both volcano plots and enrichment analyses were performed in MetaboAnalyst 5.0.

Integrating vaginal microbiome profiles with VAS and VuAS surveys: To analyze the vaginal microbiome profiles between high and low VAS and VuAS groups, MicrobiomeAnalyst 2.0 was utilized. Taxa counts were filtered, as stated above, to 4 reads and 10% prevalence and then rarefied to the minimum library size. Alpha-diversity was computed using the Shannon diversity index and Chao1, and VAS group comparisons were performed with Mann-Whitney U tests. Beta-diversity metrics were computed using Bray-Curtis dissimilarity index, and VAS group comparisons were performed with the nonparametric permutational multivariate analysis of variance (PERMANOVA) and visualized with PCA plots. The relative abundances of taxa in the high and low VAS groups were visualized as stacked bar plots. Taxa were plotted at the genus level and any taxa with <10 read counts were merged into an “Others” category.

Differential abundance analysis and prediction of functional gene content: ASVs identified at the species level were analyzed utilizing Analysis of compositions of microbiomes with bias correction (ANCOM-BC) R package to determine if they were differentially abundant between the high and low VAS and VuAS groups, as well as between the presence and absence of individual VAS and VuAS symptoms. P values were corrected for multiple comparisons using the false discovery rate (FDR) method, and taxa with a log fold change (LFC)>1 and a q-value <0.05 were considered significant and graphically presented as a dot plot. Taxa related to vaginal irritation were significant at P<0.05, not after FDR correction.

To determine the potential metabolic contributions of the identified vaginal bacterial taxa, ASVs from QIIME 2 were imported into Phylogenetic Investigation of Communities by Reconstruction of Unobserved States (PICRUSt2) utilizing q2-picrust2. Host and microbial genomes from the Kyoto Encyclopedia of Genes and Genomes (KEGG) database and the PICRUSt2 predicted metagenomes were used to predict the functional gene content of the 16S rRNA profiles. ANCOM-BC was performed to determine significant metabolic pathways. Analysis was performed between the high and low VAS and VuAS groups. Significant pathways, considered at LFC>1 and P<0.05 without FDR correction, were named using MetaCyc database and graphically presented in a volcano plot.

Correlation analyses of omics and survey data: Spearman's rank correlation analysis was utilized to measure the strength and direction of associations between different omics and survey data. Spearman's rank correlation coefficients (r) were calculated between all sexual health measures (eight PROMIS SexFS measures, total VAS scores, and total VuAS scores) and displayed as a correlation matrix. The eight PROMIS SexFS measures were correlated to metabolite and immune protein levels, and total VAS scores, total VuAS scores, and individual vaginal symptoms were also correlated to immune protein levels. Significant metabolite and protein levels were correlated together, as well as relative abundances of vaginal bacterial taxa, separately. Correlation coefficients were calculated using the maximum number of patients for each variable, and critical values were determined based on each sample size. P values <0.05 were considered significant.

Other statistical analysis: Differences in patient demographics, socioeconomics, and medical history between the VAS and VuAS groups were calculated using an unpaired t-test for continuous variables and Fisher's exact test for categorical variables. Multiple testing was corrected for by false discovery rate (FDR). P values <0.05 were considered significant

Patient Characteristics: In this cross-sectional study, 108 women were enrolled at the time of surgery undergoing hysterectomy for benign gynaecological conditions. CVLs and vaginal swabs were collected for omics analyses. To assess their sexual health profiles, participants were provided with three surveys: Vaginal Assessment Scale (VAS), Vulvar Assessment Scale (VuAS), and Patient-Reported Outcome Measurement System Sexual Function and Satisfaction v2.0 Brief Profile (PROMIS SexFS). A total of 79 participants completed at least one sexual health measure to form the study sample. Participants were grouped based on total VAS scores (n=67) or total VuAS scores (n=69) into low (scores 0-2; n=38) and high VAS (score 3-12; n=29), and low (score 0; n=35) and high VuAS (scores 1-12; n=34). Group cut-offs were determined by the median total scores. The mean age and body mass index (BMI) of all patients (n=79) was 44.4 years and 29.5 kg m 2, respectively. Most patients were Caucasian (72.2%), premenopausal (87.3%), and had a normal vaginal pH (£ 4.5) (78.5%). Regarding ethnicity, 27.9% of participants identified as Hispanic. Age, BMI, race, ethnicity, menopausal status, and vaginal pH were not significantly different between VAS or VuAS groups (Table 2). Since age and menopausal status are key factors for the mucosal environment, additional multiple linear regression analyses were performed, confirming that age and menopausal status did not significantly differ between groups (VAS: P=0.1823; P=0.1104; VuAS: P=0.3027; P=0.3120, respectively). More women with high VAS scores self-reported having chronic pelvic pain (CPP) and endometriosis history compared to women with low VAS scores (84.6% vs 43.3%; P=0.0002, and 48.3% vs 19.4%; P=0.0176). Polycystic ovary syndrome, diabetes, and hypertension were not significantly different between groups. Detailed patient demographics and characteristics are included in.

TABLE 2
Patient demographics, characteristics, and medical history between low and high total 313 VAS and VuAS groups.

| | | | Patients grouped by total VAS score | | | | | Patients grouped by total VuAS score | | | | |
|---|---|---|---|---|---|---|---|---|---|---|---|---|
| | All | | Low VAS | | High VAS | | P value | Low VuAS | | High VuAS | | P value |
| Sample size | N = 79 | | n = 38 | | n = 29 | | | n = 35 | | n = 34 | | |
| Age [mean (SD)] | 44.43 | (9.68) | 45.95 | (10.82) | 42.62 | (8.83) | 0.2872 | 45.40 | (10.41) | 43.03 | (8.41) | 0.2848 |

Race [n (%)]

| | | | | | | | | | | | | |
|---|---|---|---|---|---|---|---|---|---|---|---|---|
| White/Caucasian | 57 | (72.15) | 30 | (78.95) | 18 | (62.07) | 0.1427 | 26 | (74.29) | 24 | (70.59) | 0.6458 |
| Black/African American | 8 | (10.13) | 5 | (13.16) | 2 | (6.90) | | 4 | (11.43) | 3 | (8.82) | |
| American Indian/Alaska Native | 3 | (3.80) | 0 | (0.0) | 3 | (10.34) | | 0 | (0.00) | 3 | (8.82) | |
| Asian/Far East/Southeast | 4 | (5.06) | 1 | (2.63) | 2 | (6.90) | | 1 | (2.86) | 2 | (5.88) | |
| Mixed or Multiracial | 6 | (7.59) | 2 | (5.26) | 3 | (10.34) | | 3 | (8.57) | 2 | (5.88) | |
| Other | 1 | (1.27) | 0 | (0.00) | 0 | (0.00) | | 1 | (2.86) | 0 | (0.00) | |

Ethnicity [n (%)]

| | | | | | | | | | | | | |
|---|---|---|---|---|---|---|---|---|---|---|---|---|
| Non-Hispanic | 57 | (72.15) | 23 | (73.68) | 19 | (65.52) | 0.5916 | 24 | (66.57) | 25 | 73.53) | 0.7918 |
| Hispanic | 22 | (27.55) | 10 | (26.32) | 10 | (34.48) | | 11 | (31.43) | 9 | (26.47) | |
| BMI [mean (SD)] | 29.55 | (7.13) | 30.04 | (8.81) | 29.57 | (5.58) | 0.8036 | 30.25 | (8.99) | 29.08 | (5.73) | 0.6233 |

Menopausal status [n (%)]

| | | | | | | | | | | | | |
|---|---|---|---|---|---|---|---|---|---|---|---|---|
| Premenopausal | 69 | (87.34) | 30 | (78.95) | 27 | (93.10) | 0.1679 | 29 | (82.86) | 31 | (91.16) | 0.4773 |
| Postmenopausal | 10 | (12.66) | 8 | (21.05) | 2 | (6.90) | | 6 | (17.14) | 3 | (6.82) | |

Benign gynaecological condition diagnosis [n (%)]

| | | | | | | | | | | | | |
|---|---|---|---|---|---|---|---|---|---|---|---|---|
| Adenomyosis | 35 | (44.30) | 17 | (44.74) | 14 | (48.28) | 0.8089 | 17 | (48.57) | 13 | (38.24) | 0.4691 |
| Endometriosis | 13 | (16.45) | 8 | (21.05) | 4 | (13.79) | 0.5314 | 7 | (20.00) | 5 | (17.65) | 1 |
| Fibroids | 49 | (62.03) | 23 | (60.53) | 16 | (51.72) | 0.6192 | 23 | (65.71) | 17 | (50.00) | 0.227 |
| None | 13 | (16.45) | 6 | (15.79) | 6 | (20.69) | 0.7501 | 3 | (8.57) | 10 | (29.41) | 0.0340 |

Vaginal pH [n (%)]

| | | | | | | | | | | | | |
|---|---|---|---|---|---|---|---|---|---|---|---|---|
| ≤4.5 | 62 | (78.48) | 28 | (73.58) | 23 | (79.31) | 0.7736 | 26 | (74.29) | 27 | (79.41) | 0.7766 |
| >4.5 | 17 | (21.52) | 10 | (26.32) | 6 | (20.69) | | 9 | (25.71) | 7 | (20.59) | |

Chronic pelvic pain history [n (%)]

| | | | | | | | | | | | | |
|---|---|---|---|---|---|---|---|---|---|---|---|---|
| No | 31 | (41.89) | 21 | (56.76) | 4 | (15.38) | 0.0002 | 18 | (54.55) | 10 | (31.25) | 0.0804 |
| Yes | 43 | (58.11) | 16 | (43.24) | 22 | (84.62) | | 15 | (45.45) | 22 | (66.75) | |

Endometriosis history [n (%)]

| | | | | | | | | | | | | |
|---|---|---|---|---|---|---|---|---|---|---|---|---|
| No | 54 | (70.13) | 29 | (80.56) | 15 | (51.72) | 0.0176 | 28 | (78.79) | 21 | (61.75) | 0.1826 |
| Yes | 23 | (29.57) | 7 | (19.44) | 14 | (48.28) | | 7 | (21.21) | 13 | (38.24) | |

PCOS history [n (%)]

| | | | | | | | | | | | | |
|---|---|---|---|---|---|---|---|---|---|---|---|---|
| No | 63 | (87.50) | 32 | (94.12) | 21 | (77.78) | 0.1229 | 29 | (90.63) | 24 | (60.00) | 0.2940 |
| Yes | 9 | (12.50) | 2 | (5.88) | 6 | (22.22) | | 3 | (9.38) | 6 | (20.00) | |

Diabetes [n (%)]

| | | | | | | | | | | | | |
|---|---|---|---|---|---|---|---|---|---|---|---|---|
| No | 65 | (82.28) | 33 | (86.84) | 21 | (72.41) | 0.2124 | 29 | (82.86) | 27 | (79.41) | 0.7660 |
| Yes | 14 | (17.72) | 5 | (13.16) | 6 | (27.59) | | 6 | (17.14) | 7 | (20.59) | |

Hypertension [n (%)]

| | | | | | | | | | | | | |
|---|---|---|---|---|---|---|---|---|---|---|---|---|
| No | 63 | (79.75) | 32 | (84.21) | 22 | (75.86) | 0.5346 | 28 | (80.00) | 28 | (82.35) | 1 |
| Yes | 16 | (20.25) | 6 | (15.79) | 7 | (24.14) | | 7 | (20.00) | 6 | (17.65) | |

Sexual health measure correlation matrix: To analyze the relationships between self-reported vulvovaginal symptoms and sexual health measurements, Spearman's correlation coefficients (r) were calculated, and unsupervised hierarchical clustering was performed between the eight PROMIS SexFS domains and total VAS and VuAS scores (FIG. 1). The correlation matrix with dendrogram revealed two distinct clusters of sexual health measurements that significantly correlated to each other (P ranging from 0.005 to <0.0001; r ranging from 0.360 to 0.789). Cluster 1 is composed of all measures related to positive sexual health outcomes (such as orgasm ability, orgasm pleasure, sexual interest, sexual satisfaction, and vaginal lubrication), and Cluster 2 is composed of all measures related to negative sexual health outcomes (such as vaginal discomfort, vulvar discomfort-labial, vulvar discomfort-clitoral, total VAS and total VuAS). Significant negative correlations were observed between orgasm ability, orgasm pleasure, and vaginal lubrication with vulvovaginal discomfort measures (P ranging from 0.04 to 0.001; r ranging from −0.273 to −0.414). On the other hand, sexual satisfaction and sexual interest were not significantly correlated with any vulvovaginal discomfort measures. Overall, this demonstrates that women with more severe vulvovaginal symptoms report lower orgasm pleasure, orgasm ability, and vaginal lubrication, but sexual satisfaction and interest are independent of vulvovaginal symptom severity.

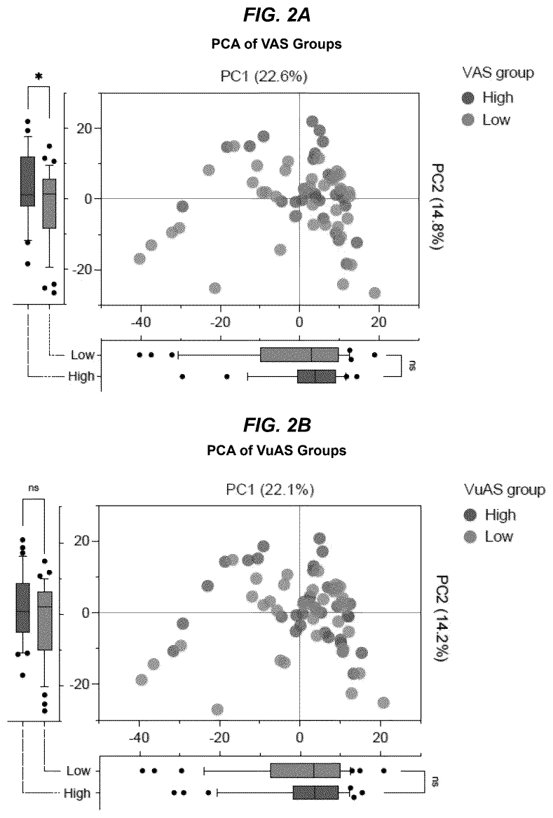

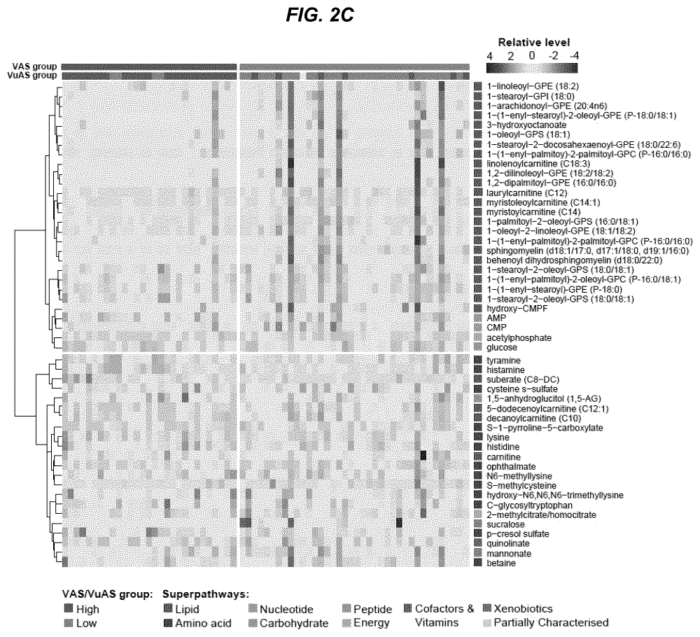

Global metabolic profiles: CVL samples were collected from all patients (n=79) and used for untargeted global metabolomics analysis. 800 metabolites of known identity across nine metabolite superpathways were identified and quantified, including 228 lipids (29%), 206 amino acids (26%), 172 xenobiotics (22%), 67 nucleotides (8%), 40 peptides (5%), 34 carbohydrates (4%), 32 cofactors and vitamins (4%), 12 energy (2%), and 9 partially characterized metabolites (1%). Principal component analysis (PCA) was used to depict the global metabolic profiles of patients stratified based on VAS and VuAS groupings (FIGS. 2A and 2B, respectively). The first two principal components (PC1 and PC2) explained 37.4% and 36.3% of the variance in the data for VAS and VuAS, respectively. Pairwise comparisons revealed PC2, but not PC1, significantly differed between low and high VAS groups (P=0.049), suggesting differences in the global profiles between these groups. PC1 and PC2 did not significantly differ between low and high VuAS groups.

Hierarchical clustering analysis was performed to analyze global metabolic profiles further. The top 50 significant metabolites between the low and high VAS groups were determined by t-test and depicted as a heatmap (FIG. 2C). Unsupervised clustering of metabolites revealed two distinct clusters; Cluster 1 composed predominantly of lipid metabolites (92%), and Cluster 2 of mostly amino acid metabolites (64%). Supervised clustering of samples by VAS scores revealed that women with low VAS scores had higher relative levels of these lipid and amino acid metabolites than those with high VAS scores. In summary, these analyses revealed significant differences in the global metabolic profiles, particularly in lipid and amino acid metabolism, between VAS groups and for VuAS groups.

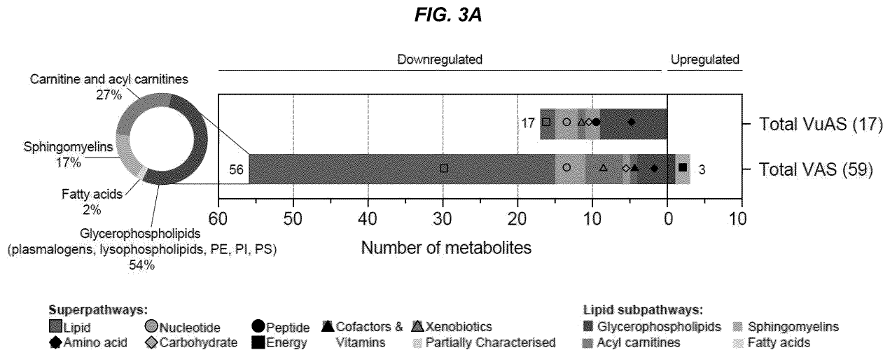

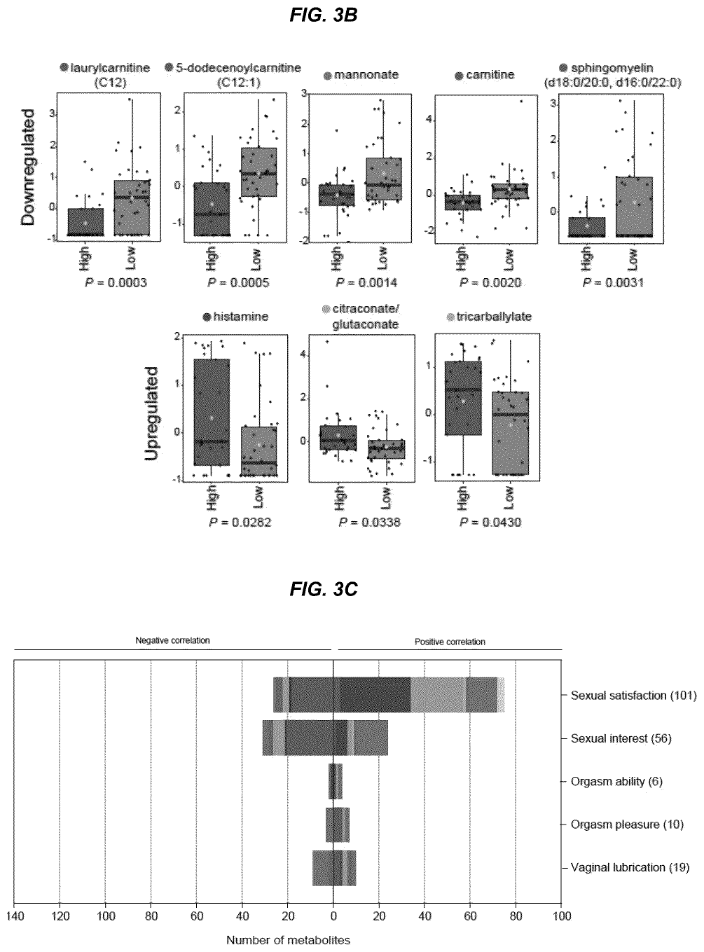

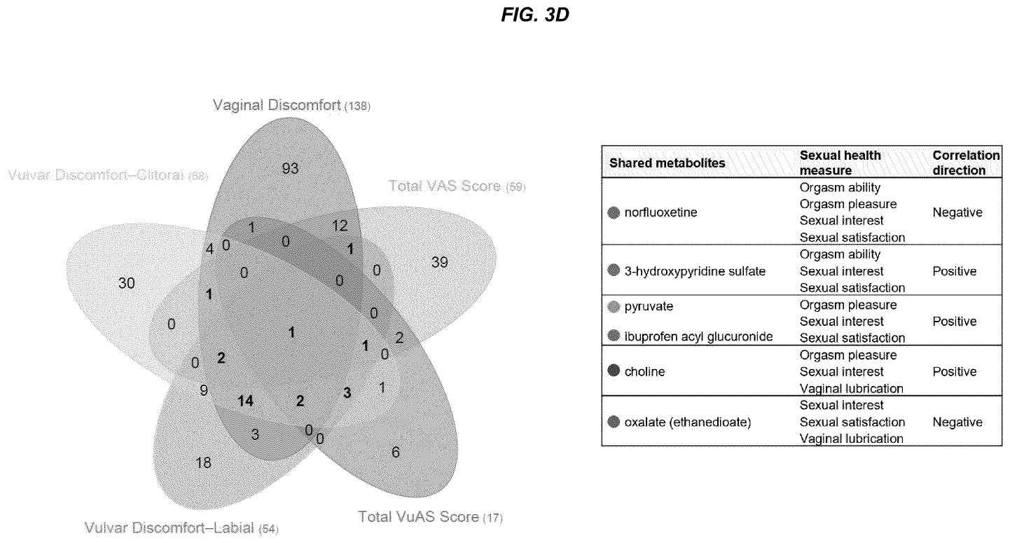

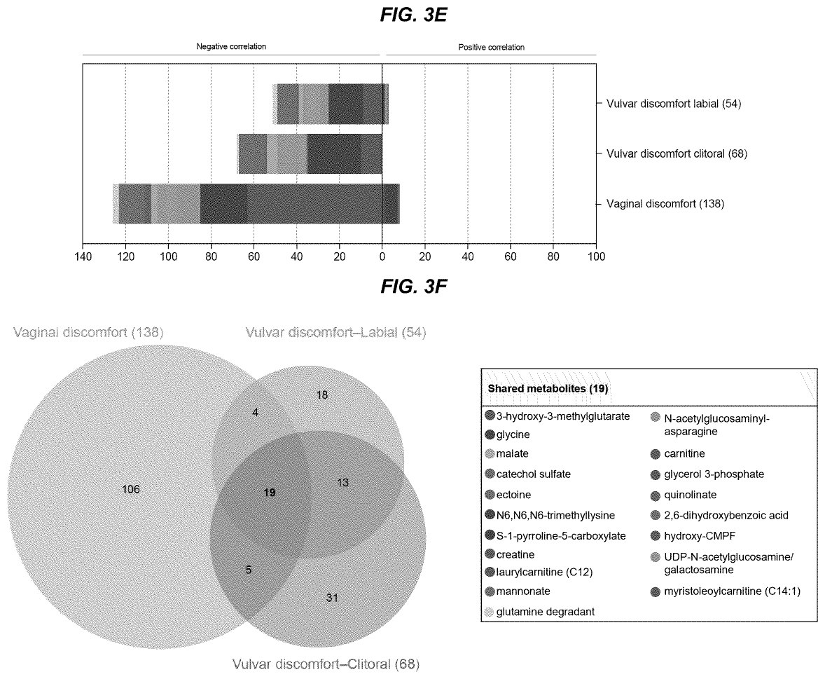

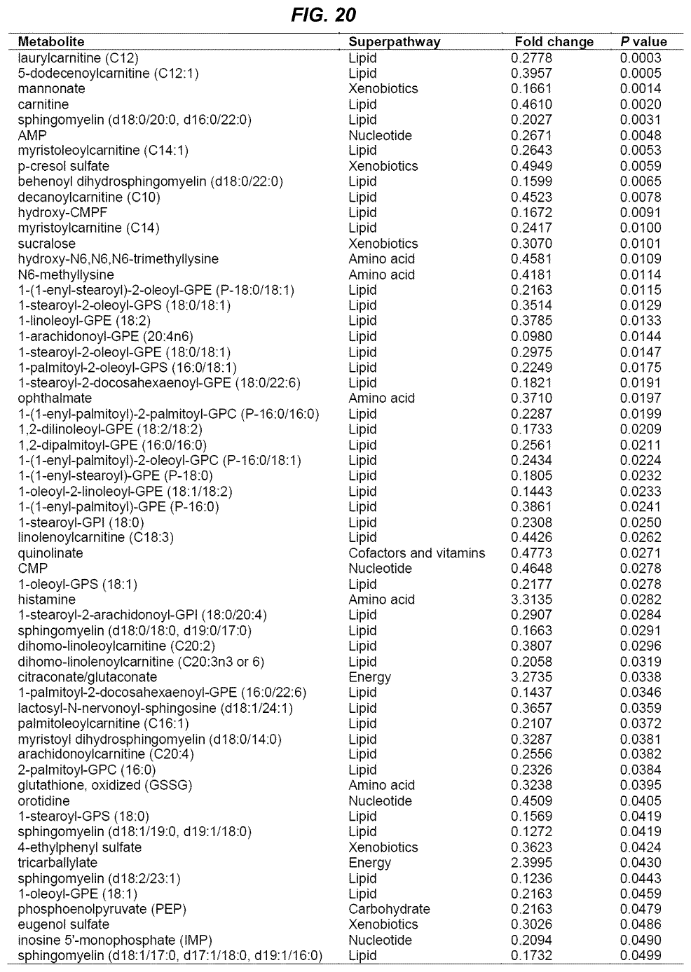

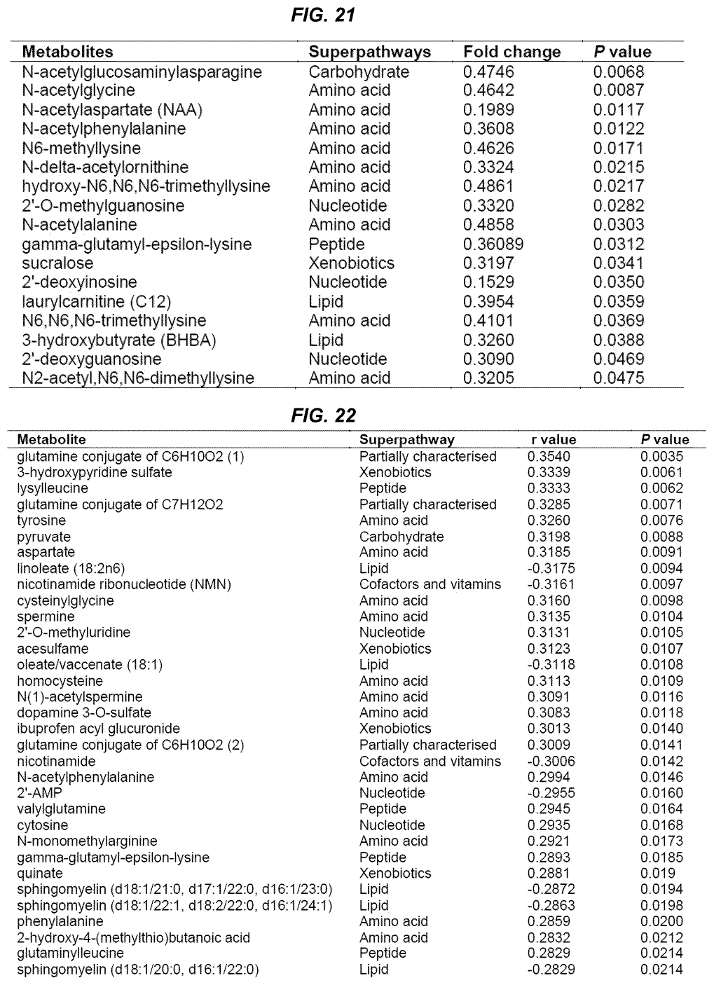

Altered lipid and amino acid metabolites associated with sexual health measures: Next, the significantly altered metabolites between VAS and VuAS groups using two-sample t-tests and fold change (FC) analysis (P value <0.05; FC>2) was determined (FIG. 20 and FIG. 21). This revealed 17 significantly altered metabolites in high vs. low VuAS groups, all downregulated, and 59 significantly altered metabolites in high vs. low VAS groups, 56 downregulated and 3 upregulated. These metabolites were then categorized by their superpathway (FIG. 3A). Women with higher VuAS scores had mostly downregulation of amino acids (53%), whereas for high VAS scores, there was mostly a downregulation of lipids (69%), followed by xenobiotics (8%), amino acids (7%), nucleotides (7%), carbohydrates (2%), and cofactors and vitamins (2%), with one amino acid (2%) and two energy metabolites (3%) upregulated. These downregulated lipids consisted of glycerophospholipids (53%; including lysophospholipids, phosphatidylethanolamines (PE), phosphatidylinositols (PI), phosphatidylserines (PS), and plasmalogens), acylcarnitines (27%), sphingomyelins (17%), and fatty acids (2%), most of which are essential cell membrane structural components. The five most significantly downregulated metabolites in the high VAS group were predominately related to lipid metabolism-laurylcarnitine (C12) (P=0.0003), 5-dodecenoylcarnitine (C12: 1) (P=0.0005), carnitine (P=0.0020), and sphingomyelin (d18:0/20:0, d16:0/22:0) (P=0.0031)—and one xenobiotic, mannonate (P=0.0014) (FIG. 3B). The three significantly upregulated metabolites were histamine (P=0.0282), citraconate/glutaconate (P=0.0338), and tricarballylate (P=0.0430).

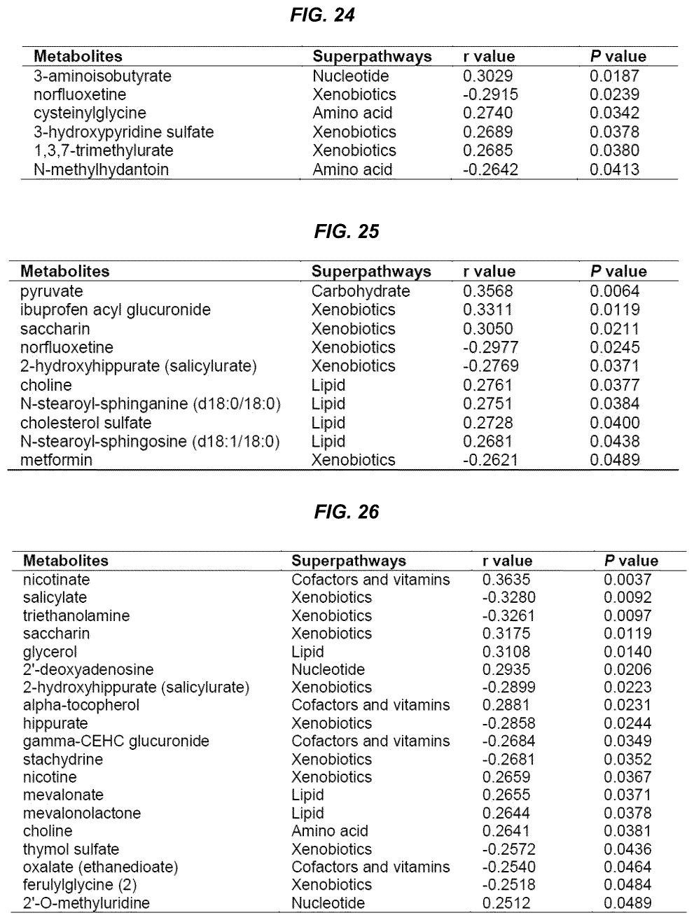

In addition, Spearman correlation analysis was performed to explore the relationship between the eight PROMIS SexFS measures and metabolite levels. Amongst the positive sexual health measures, vastly different metabolite signatures were performed (FIG. 3C; FIG. 22, FIG. 23, FIG. 24, FIG. 25 and FIG. 26). Sexual satisfaction and sexual interest were associated with 101 and 56 metabolites, respectively; far more than for orgasm ability, orgasm pleasure, and vaginal lubrication, which significantly correlated with 6, 10, and 19 metabolites, respectively. Despite sexual satisfaction and interest negatively correlating with mostly lipids (69% and 65%, respectively), only two were shared with lipids altered in the high VAS group. Of the metabolites that positively correlated with sexual satisfaction, 41% were amino acids, and 25% were peptides, including N-acetylated and essential amino acids, aromatic lactic acids, and dipeptides. Orgasm ability, orgasm pleasure, and vaginal lubrication significantly correlated with mostly xenobiotics: 50%, 50%, and 47%, respectively. Most metabolites were unique to each positive sexual health measure (FIG. 3D). Sexual satisfaction and sexual interest shared the greatest number of metabolites (23). Six of these shared metabolites were mostly xenobiotics and correlated with other variables: norfluoxetine, an antidepressant metabolite, negatively correlated with orgasm pleasure and orgasm ability, oxalate negatively correlated with vaginal lubrication, 3-hydroxypyridine sulfate positively correlated with orgasm ability, choline positively correlated with vaginal lubrication, and pyruvate and ibuprofen acyl glucuronide positively correlated with orgasm pleasure. Another antidepressant metabolite, O-desmethylvenlafaxine, was also negatively associated with sexual interest and vaginal lubrication.

Vaginal discomfort negatively correlated with mostly lipids (50%), similarly to VAS, and vulvar discomfort-clitoral and labial negatively correlated with mostly amino acids (37% and 31%, respectively) (FIG. 3E; FIG. 27, FIG. 28, and FIG. 29), similarly to VuAS. Vaginal, clitoral, and labial discomfort shared only 7% of the identified metabolites (19/260). Vaginal discomfort had the greatest number of unique metabolites (106), whereas clitoral and labial discomfort shared the most metabolites (32) (FIG. 3F). Some of the shared metabolites were also altered in the high VAS group; laurylcarnitine (C12), mannonate, carnitine, quinolinate, hydroxy-CMPF, and myristoleoylcarnitine (C14:1). This analysis revealed distinct metabolic signatures between positive and negative sexual health measures and from those of VAS and VuAS scores.

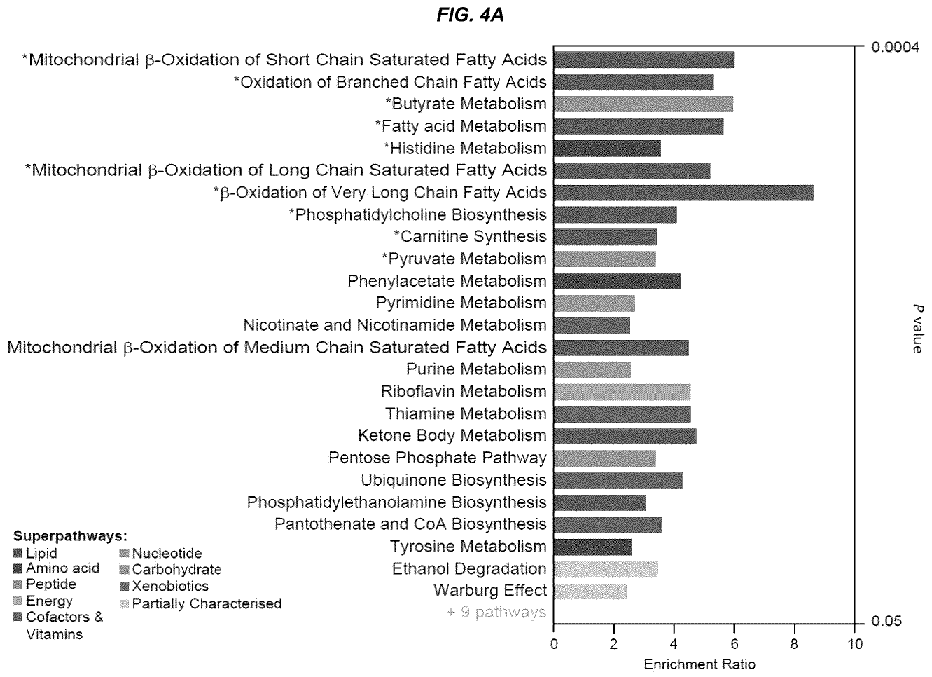

Dysregulated metabolic pathways: Pathway analysis was then performed to identify perturbed metabolic pathways between women with high and low VAS (FIG. 4A) and VuAS scores (FIG. 4B). This revealed 34 significantly (P<0.05) dysregulated pathways between VAS groups, 10 of which were significant after FDR correction (q<0.05). The most significant dysregulated pathways between VAS groups were associated with phospholipid and fatty acid metabolism (n=7; mitochondrial beta-oxidation of short-chain saturated fatty acids, oxidation of branched-chain fatty acids, fatty acid metabolism, mitochondrial beta-oxidation of long-chain saturated fatty acids, beta-oxidation of very long chain fatty acids, phosphatidylcholine biosynthesis, and carnitine synthesis), nucleotide metabolism (n=2; butyrate and pyruvate), and amino acid metabolism (n=1, histidine). Ubiquinone biosynthesis was significantly altered in VuAS and VAS groups, although not following FDR correction.

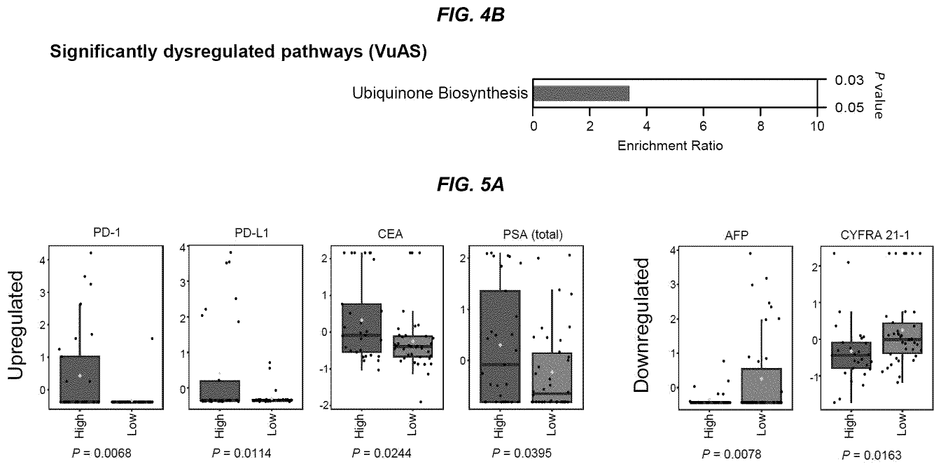

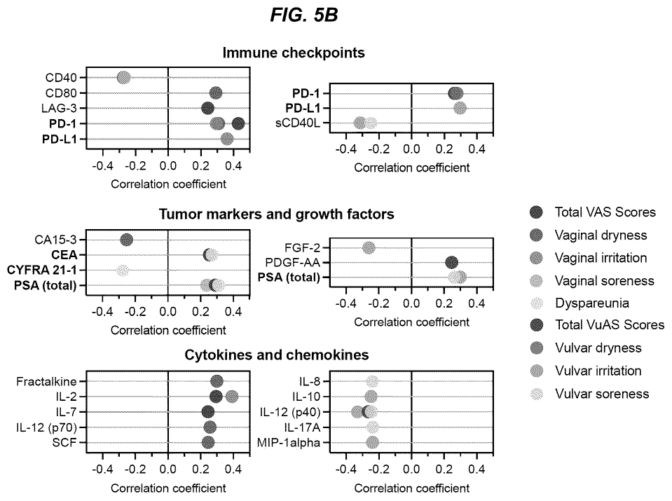

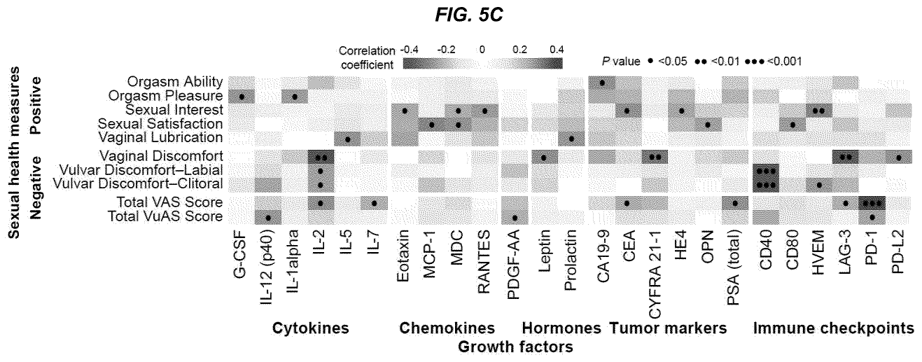

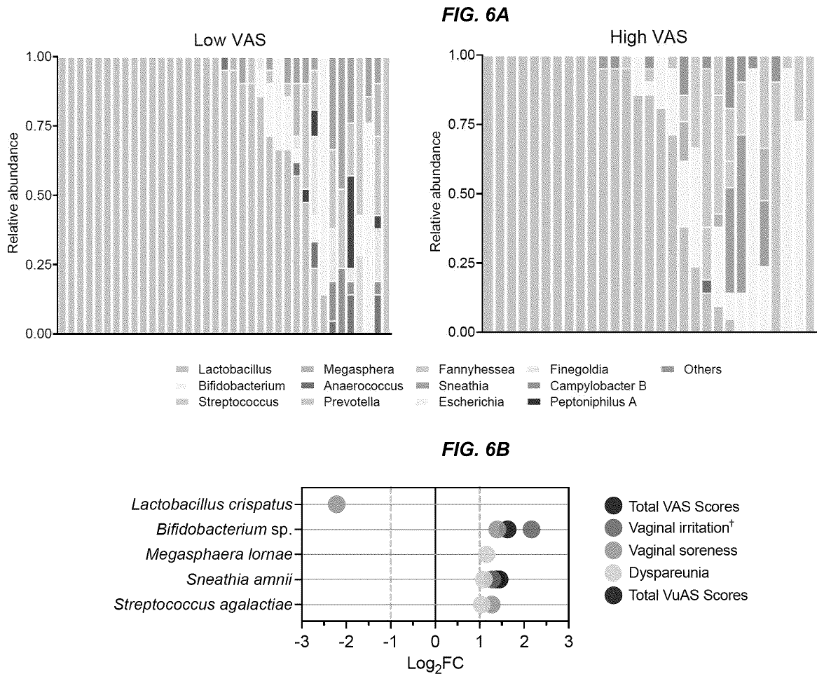

Altered cervicovaginal immune proteins associated with sexual health measures: To analyze the immunoproteomic profiles of the cohort, levels of 72 soluble proteins from CVL samples were quantified, including cytokines, chemokines, growth factors, hormones, circulating tumour markers, apoptosis-related proteins, and immune checkpoint proteins. Significantly altered proteins were determined between the VAS and VuAS groups using a two-sample t-test and FC analysis (P value <0.05; FC>2). No significance was observed following FDR correction (q<0.05). The immune checkpoint protein programmed cell death ligand 1 (PD-L1) was significantly upregulated among women with high VuAS scores and high VAS scores (FIG. 5A). Its receptor programmed cell death protein 1 (PD-1) (P=0.0068) was also significantly upregulated in women with high VAS scores, along with tumour markers, carcinoembryonic antigen (CEA) (P=0.0244) and prostate-specific antigen (PSA (total)) (P=0.0395). Other tumour markers, cytokeratin 19 fragment (CYFRA 21-1) (P=0.0163) and alpha-fetoprotein (AFP) (P=0.0078), were significantly upregulated in patients with higher VAS scores.

Next, Spearman correlation analysis was utilized to identify proteins associated with individual vaginal and vulvar symptoms. PD-1, PD-L1, CEA, and PSA (total) significantly and positively correlated with many individual vulvovaginal symptoms (P ranging from 0.043 to 0.0003) (FIG. 5B). In addition, cluster of differentiation (CD) 80 and lymphocyte activating gene 3 (LAG-3) significantly and positively correlated with vaginal dryness and total VAS score, respectively, and CD40 significantly and negatively correlated with vaginal soreness and dryness. Multiple cytokines and chemokines significantly and positively correlated with vaginal symptoms: fractalkine, interleukin (IL)-2, IL-7, IL-12 (p70), and stem cell factor (SCF) (P ranging from 0.045 to 0.001). Immune mediators also significantly and negatively correlated with vulvar symptoms: IL-8, IL-10, IL-12 (p40), IL-17A, and macrophage inflammatory protein (MIP)-1a (P ranging from 0.049 to 0.006).

Many immune proteins were also associated with the positive and negative PROMIS SexFS measures, identified by Spearman correlation (FIG. 5C). Orgasm ability was significantly and positively correlated with tumour marker, carbohydrate antigen (CA) 19-9, and orgasm pleasure with cytokines, granulocyte colony-stimulating factor (G-CSF), and IL-1a (P ranging from 0.04 to 0.024). Sexual satisfaction and interest negatively correlated with chemokines including eotaxin, monocyte chemoattractant protein (MCP)-1, macrophage-derived chemokine (MDC), and regulated on activation, normal T cell expressed and secreted (RANTES) (P ranging from 0.045 to 0.014). Vaginal lubrication correlated negatively with IL-5 (P=0.03) and a hormone, prolactin (P=0.038). All three vulvovaginal discomfort measures significantly and positively correlated with IL-2 (P ranging from 0.039 to 0.003), and both vulvar discomfort measures significantly negatively correlated with CD40 (P<0.001; r<−0.4). Overall, the levels of immune proteins in cervicovaginal lavages significantly correlate with sexual health measures, and this large immunoregulatory signature was observed in patients with more severe vulvovaginal discomfort symptoms.

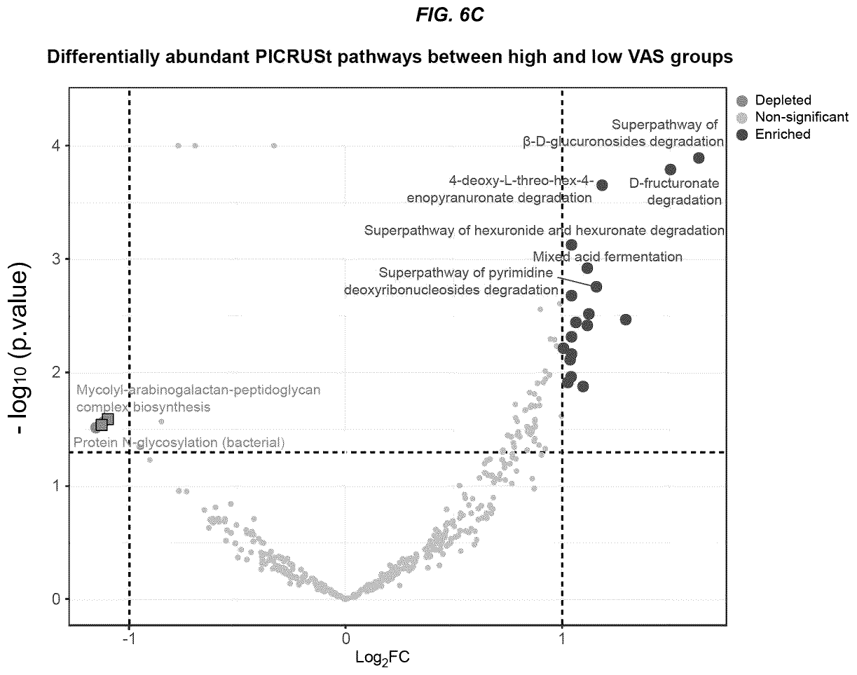

Vaginal microbiome profiles and their potential microbial metabolic contributions: Next, the relationship between vaginal microbiota composition and vulvovaginal symptoms was assessed. The overall profiles of this cohort displayed a prevalence of Lactobacillus. Between high and low VAS groups, the overall vaginal microbiota composition did not differ (FIG. 6A). Microbiome diversity metrics, including Shannon and Chao1 α-diversity indexes and Bray-Curtis's b-diversity, were not significantly different between high and low VAS and VuAS groups. Lactobacillus dominance (defined as the relative abundance of >80%), which is associated with vaginal health, was also not significantly different between the high and low VAS groups (45% vs. 57%, respectively; P=0.4514).

The differential abundance of species was then analyzed between high and low VAS and VuAS scores, as well as the presence or absence of individual vulvovaginal symptoms (FIG. 6B). Lactobacillus crispatus, which is considered optimal for vaginal health, was significantly (q<0.05) depleted in women experiencing vaginal soreness, whereas dysbiotic bacteria-Sneathia amnii (recently renamed Sneathia vaginalis), Megasphaera lornae (also previously known as Megasphaera type 1), Streptococcus agalactiae (also known as Group B Streptococcus), and Bifidobacterium sp.—were significantly (q<0.05) enriched in women with more severe vaginal symptoms, including soreness, irritation, dyspareunia, and total VAS score, although irritation was not significant after FDR correction (P<0.05). S. amnii was also identified to be significantly (q<0.05) enriched in women with high VuAS scores. Although global microbiome profiles did not significantly differ between groups, this analysis identified altered levels of specific dysbiotic vaginal bacteria in women with more severe vulvovaginal symptoms.

To analyze the potential metabolic pathway contributions of the vaginal microbiota, PICRUSt pathway analysis was performed. This identified 20 significantly altered pathways based on the microbiota composition between high and low VAS groups (FIG. 6C). Eighteen pathways were upregulated, including 3-D-glucuronoside degradation (P=<0.0001), D-fructuronate degradation (P=0.0001), and 4-deoxy-L-threo-hex-4-enopyranuronate degradation (P=0.0001), and two pathways were downregulated: mycolyl-arabinogalactan-peptidoglycan complex biosynthesis (P=0.029) and bacterial protein N-glycosylation (P=0.03). These results indicated the involvement that many bacterial pathways may have in perturbed metabolic pathways related to carbohydrate, amino acid, and nucleotide metabolism in women suffering from vulvovaginal symptoms.

Integration of metabolome, immunoproteome, and microbiome profiles: To better understand the interactions between the microbiome and the identified significant metabolites and immune proteins that related to vulvovaginal and sexual health measures, Spearman correlation analysis was performed between these omics and displayed them as heatmaps with unsupervised clustering.

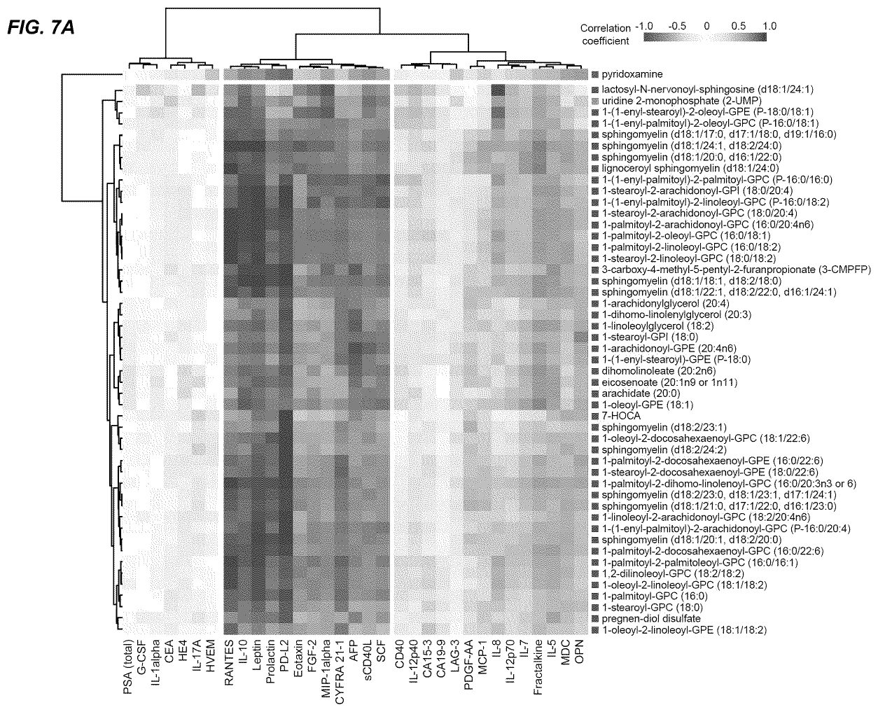

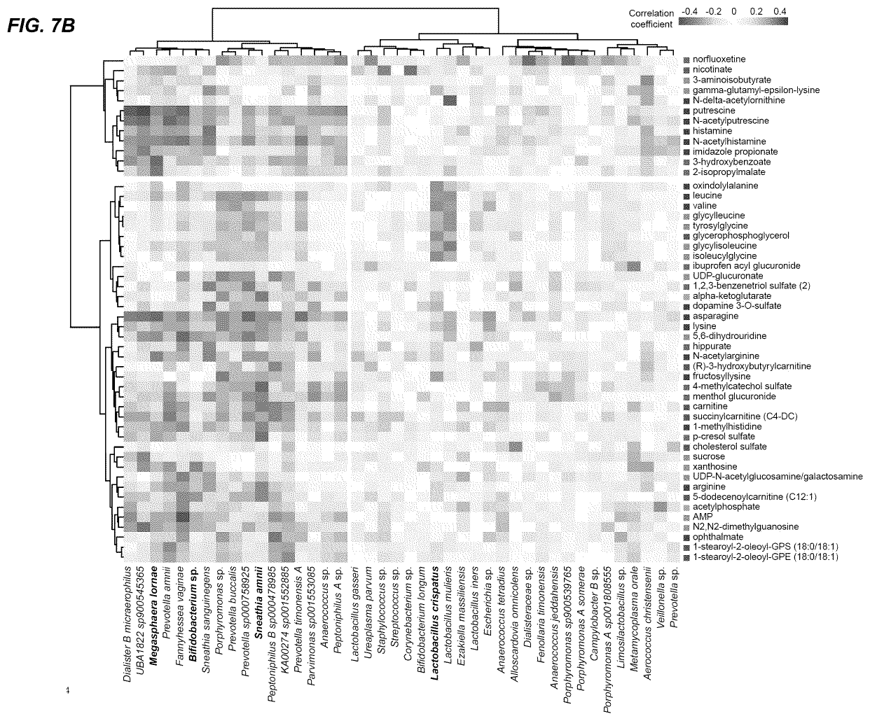

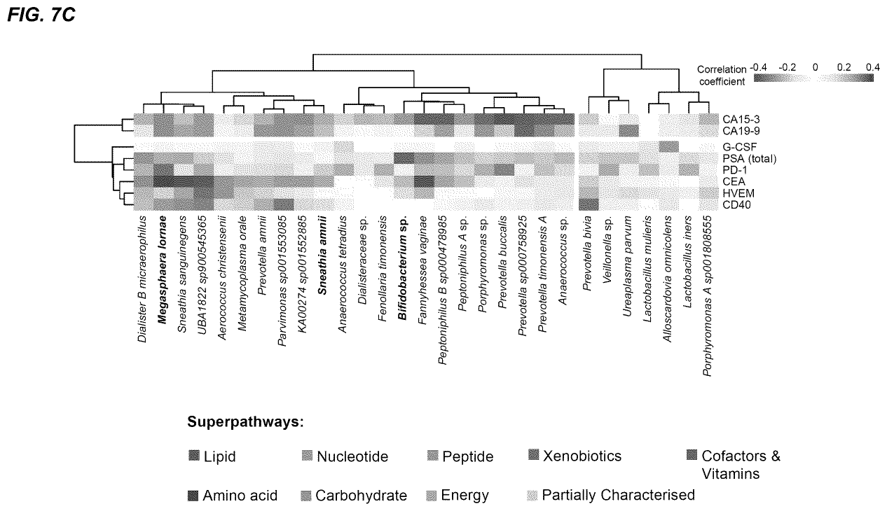

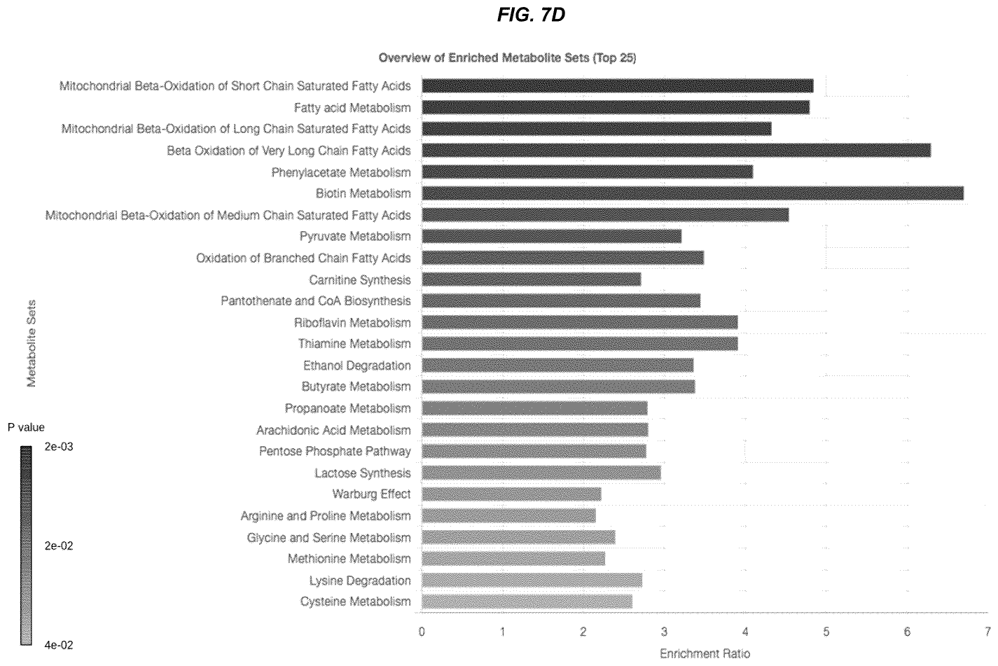

Of the top 50 strongest correlating metabolites with immune proteins, lipid-related metabolites were the most prominent (48/50) (FIG. 7A). These lipids, along with one nucleotide-related metabolite, uridine 2-monophosphate (2-UMP), significantly positively correlated with numerous altered immune proteins, including chemokines: RANTES, eotaxin, and MIP1-a; cytokines: IL-10 and SCF; hormones: leptin and prolactin; immune checkpoint proteins: PD-L2 and sCD40L; tumour markers: CRYFRA 21-1 and AFP; and growth factor, FGF-2. Many of these lipids and immune proteins had negatively correlated with high sexual interest, sexual satisfaction, and vaginal discomfort, or were downregulated in high VAS. In contrast, the vitamin B6 derivative, pyridoxamine, strongly negatively correlated with these immune proteins and had previously positively correlated with sexual satisfaction.

Next, metabolites were correlated with vaginal bacteria and observed weaker correlations than previously observed between metabolites and immune proteins (r ranging from −0.5052 to 0.4704 vs. −0.6926 to 0.8491) (FIG. 7B). Numerous amino acids, including the biogenic amines putrescine and N-acetylputrescine, and three histidine derivatives, histamine, N-acetylhistamine, and imidazole propionate, were positively correlated with multiple dysbiosis-associated vaginal species including M. lornae, Prevotella amnii, Fannyhessea vaginae, Sneathia sanguinegens, Prevotella sp., Bifidobacterium sp., and UBA1822 sp900545365 (Dialisteraceae family). These metabolites had positively associated with vaginal discomfort, linking these other dysbiotic bacteria to symptoms of discomfort. These species, along with S. amnii, negatively correlated with other amino acids, including asparagine, lysine, N-acetylarginine, and putrescine precursor arginine, all of which positively associated with sexual satisfaction. Many acyl carnitines that were negatively associated with vaginal discomfort also negatively correlated with these dysbiotic vaginal species. Lactobacillus mulieris and L. crispatus positively correlated with sexual satisfaction-associated metabolites: valine, leucine, glycylleucine, glycylisoleucine, isoleucylglycine, tyrosylglycine, and glycerophosphoglycerol. These metabolites also negatively correlated with Prevotella spp. (P. buccalis, P. timonensis A, Prevotella sp000758925), Porphyromonas sp., and S. amnii. Xenobiotics also had significant correlations with microbes, including norfluoxetine with Dialisterceae sp. and Porphyromonas sp900539765, and ibuprofen acyl glucuronide with Metamycoplasma orale and Ureaplasma parvum.

Correlation analysis between immune proteins and vaginal bacteria revealed eight immune proteins with significantly strong correlations (FIG. 7C). CA15-3, negatively correlated with vaginal dryness, and CA19-9, positively associated with orgasm ability, both significantly negatively correlated with 12 dysbiosis-associated bacteria including from the genera Megaspheara, Prevotella, Anaeococcus, Porphyromonas, Parvimonas, Peptoniphilus, and Sneathia. In addition, the orgasm pleasure-associated cytokine, GCS-F, negatively correlated with the pathogen Alloscardovia omnicolens. Three proteins identified as upregulated in women with high VAS scores, PSA (total), PD-1, and CEA, were highly positively correlated with M. lornae, Dialister B micraerophilus, Peptoniphilus B sp000478985, F. vaginae, S. sanguinegens, and S. amnii, and interestingly, PD-1 also negatively correlated with L. iners and L. mulieris. Overall, this analysis revealed correlations between many metabolites and immune proteins, which were associated with negative sexual health and vaginal discomfort, to numerous dysbiosis-associated vaginal bacteria. This demonstrates the potential of these bacteria to negatively contribute to the altered cervicovaginal microenvironment observed in women suffering from vulvovaginal and sexual dysfunction symptoms.

### Example 2

The following are non-limiting examples of how the methods of the present invention may be used to guide treatment of vulvovaginal symptoms caused by genitourinary syndrome of menopause (GSM). It is to be understood that said examples are not intended to limit the present invention in any way. Equivalents or substitutes are within the scope of the present invention.

As described herein, the present invention provides physicians with objective guidance for optimizing patient treatment. By integrating a patient's verbal description of symptoms with biomarker-based measurements that quantify symptom severity, the invention enables more precise and individualized treatment decisions. Thus, the present invention not only enhances treatment decisions but may also help reduce medical bias, such as the underestimation of symptoms based solely on patient self-reporting.

Example 2.1: A patient presents with vulvovaginal symptoms including dryness and irritation. As part of the method of the present invention, the physician obtains a readout of relevant biomarkers, including serum estradiol levels and local vaginal indicators such as pH, lactobacilli abundance, mucins, epithelial maturation index on cytology and immune proteins including PD-L1 and TIM-3.

In some embodiments, epithelial maturation may be determined using one or more biomarkers, such as metabolites, including lipids. In certain embodiments, lipids associated with immature epithelium include, but are not limited to, sphingomyelin, phosphatidylcholine, lysophosphatidylcholine, and prostaglandins. In other embodiments, lipids associated with mature epithelium include, but are not limited to, ceramides, cholesterol esters, and phosphatidylserine.

Low estrogen levels along with increase in vaginal biomarkers (such as CCL20/MIP-3alpha, IL-6, IL-8, GM-CSF or lipids) and alter microbial composition support a diagnosis of genitourinary syndrome of menopause (GSM) and the physician guides therapy towards vaginal estrogen, a selective estrogen receptor modulators (SERM), or dehydroepiandrosterone (DHEA). Whereas, normal estrogen levels suggest non-hormonal causes, such as irritants or infection, guiding alternative interventions. By combining subjective symptom reports with objective biomarker readouts, the method enhances clinical decision-making and enables personalized treatment.

Example 2.2: A patient presents with vulvovaginal symptoms. Vaginal pH and microbiome are assessed, including epithelial thickness and microbial composition. An elevated pH with thin epithelium supports a diagnosis of GSM and guides treatment with vaginal estrogen. Alternatively, an altered microbiome with inflammation—as indicated by increased levels of immune proteins such as IL-1, SLPI, IL-6, IL-8, TNF-α, RANTES, IP-10, CCL20 (MIP-3a), MCP-1, and others, suggests that symptoms are driven by infection or inflammation, such as bacterial vaginosis, yeast infection, or aerobic vaginitis, indicates that symptoms are driven by infection or inflammation, guiding non-hormonal, targeted interventions instead of hormones. In certain embodiments, additional testing for bacterial and yeast infections may also be performed.

Example 2.3: A patient presents with vulvovaginal symptoms. Local inflammatory and immune biomarkers, including cytokines (IL-1B, IL-6, TNF-α), immune checkpoints (e.g., PD-1), and immune cell infiltration, are assessed. High inflammation, as indicated by an increase in at least two of the above mentioned biomarkers above a predetermined threshold, suggests infection, a dermatologic condition, or autoimmune vulvovaginitis, and guides a physician to treat with antifungal, steroid, or immune-modulating therapies. Whereas a low inflammation, as indicated by a decrease in at least two of the above mentioned biomarkers below predetermined threshold, combined with epithelial thinning is more consistent with GSM, supporting hormonal intervention.

Example 2.4: A patient presents with vulvovaginal symptoms. Tissue integrity is assessed via epithelial thickness (see biomarkers listed in Example 2.1), collagen and elastin markers, or metabolic profiles from vaginal swabs. Structural atrophy supports a diagnosis of GSM and guides a physician to treat with estrogen. Preserved tissue despite ongoing soreness suggests vulvodynia, neuropathic pain, or pelvic floor dysfunction, guiding a physician to treat with neuromodulators or recommend pelvic physical therapy.

Example 2.5: A patient presents with vulvovaginal symptoms. Pain mechanisms are assessed by measuring sensory nerve fiber density or neuroinflammatory markers, such as histamine. If pain is determined to be neurogenic rather than estrogen-deficient, treatment is guided toward neuromodulatory medications, not just estrogen therapy.

Example 2.6: A patient presents with vulvovaginal pain and dyspareunia. Histamine levels are measured. Elevated histamine suggests conditions such as vulvodynia, interstitial cystitis/bladder pain syndrome (IC/BPS), or allergic vulvitis, guiding treatment with antihistamines as a targeted therapy to reduce pain. In contrast, normal histamine levels with evidence of estrogen deficiency are more consistent with GSM, where antihistamines are not effective and may worsen dryness, guiding the physician instead toward hormonal therapy.

In summary, the present invention moves beyond trial-and-error approaches with lubricants, antifungals, or hormones by enabling clinicians to tailor therapy based on objective biomarker profiles. For example, atrophy or low-estrogen markers, including shifts in vaginal bacteria, guide treatment with estrogen therapy. An infectious or inflammatory profile directs the use of antimicrobials or anti-inflammatory agents. Neurogenic pain markers, such as histamine, indicate the need for neuromodulators or pelvic floor therapy.

### Embodiments

The following embodiments are intended to be illustrative only and not to be limiting in any way.

Embodiment 1: A method for providing a therapeutic solution to treat vulvovaginal symptoms in a female patient in need thereof, said method comprises: a) determining the patient's level of two or more biomarkers by: i) obtaining a biological sample from the patient, wherein the biological sample comprises a cervicovaginal lavage (CVL) sample or a vaginal swab; and ii) measuring the levels of at least two or more biomarkers in the biological sample obtained in (i); b) applying results from (a) to predetermined thresholds, wherein deviation of at least two biomarkers from the predetermined thresholds identifies at least one therapeutic solution for treating vulvovaginal symptoms; and c) providing the therapeutic solution to a medical professional to determine treatment for the patient.

Embodiment 2: The method of embodiment 1, wherein the vulvovaginal symptoms are caused by genitourinary syndrome of menopause (GSM). Embodiment 3: The method of embodiment 2, wherein the vulvovaginal symptoms comprise vaginal dryness, vaginal irritation, vaginal soreness, dyspareunia, vulvar dryness, vulvar irritation, vulvar soreness, and pain.

Embodiment 4: The method of embodiment 1, further comprising measuring the levels of at least five or more biomarkers. Embodiment 5: The method of embodiment 1, further comprising measuring the levels of at least ten or more biomarkers. Embodiment 6: The method of embodiment 1, further comprising measuring the levels of at least fifteen or more biomarkers.

Embodiment 7: The method of embodiment 1, wherein the two or more biomarkers comprise immune proteins, metabolites, bacterial abundance, or a combination thereof.

Embodiment 8: The method of embodiment 7, wherein the immune proteins comprise one or a combination of programmed cell death protein 1 (PD-1), programmed death-ligand 1 (PD-L1), interleukin-2 (IL-2), IL-7, alpha-fetoprotein (AFP), CYFRA 21-1 (cytokeratin 19 fragment), prostate-specific antigen (PSA), and/or carcinoembryonic antigen (CEA).

Embodiment 9: The method of embodiment 7, wherein the metabolites comprise one or more of: (i) lipids selected from glycerophospholipids, fatty acids, and sphingolipids; (ii) xenobiotics; or (iii) a combination thereof.

Embodiment 10: The method of embodiment 7 or embodiment 9, wherein the metabolites comprise one or a combination of laurylcarnitine (C12), 5-dodecenoylcarnitine, carnitine, myristoleoylcarnitine (C14:1), decanoylcarnitine (C10), mannonate, tricarballyate, citraconate/glutaconate, N-acetylglucosaminylasparagine, N-acetylglycine, N-acetylaspartate (NAA), N-acetylphenylalanine, N-delta-acetylornithine, N5-methyllysine, hydroxy-N6,N6,N6-trimethyllysine, N-acetylalanine, and gamma-glutamyl-epsilon-lysine, adenosine monophosphate (AMP) and 2′O-methylguanosine, p-cresol sulfate; and histamine.

Embodiment 11: The method of embodiment 9, wherein the lipids comprise one or a combination of behenoyl dihydrosphingomyelin (d18:0/22:0), sphingomyelin (d18:0/20:0, d16:0/22:0), 1-stearoyl-GPS (18:0), 1-palmitoyl-2-docosahexaenoyl-GPE (16:0/22:6), sphingomyelin (d18:1/22:1, d18:2/22:0, d16:1/24:1), sphingomyelin (d18:2/23:0, d18:1/23:1, d17:1/24:1), and sphingomyelin (d18:1/20:1, d18:2/20:0, d18:1/21:0, d17:1/22:0, d16:1/23:0).

Embodiment 12: The method of embodiment 7, wherein the bacterial abundance of Sneathia amnii, Megasphaera lornae, Group B Streptococcus, Lactobacillus crispatus, or a combination thereof, is measured.

Embodiment 13: The method of embodiment 1, wherein at least one of the two or more biomarkers comprises histamine. Embodiment 14: The method of embodiment 1 or embodiment 13, wherein the two or more biomarkers comprise histamine and at least one carnitine or a derivative thereof; wherein the at least one carnitine derivative is selected from a group consisting of laurylcarnitine (C12), 5-dodecenoylcarnitine, myristoleoyl-carnitine (C14:1), or decanoylcarnitine (C10). Embodiment 15: The method of embodiment 14, wherein if histamine is increased compared to a predetermined threshold and the at least one carnitine or derivative thereof is decreased compared to a predetermined threshold then the therapeutic solution comprises an antihistamine.

Embodiment 16: The method of embodiment 1, wherein the two or more biomarkers comprises two or more immune proteins selected from a group comprising programmed cell death protein 1 (PD-1), programmed death-ligand 1 (PD-L1), interleukin-2 (IL-2), IL-7, IL-12, IL-8, IL-10, Tumor Necrosis Factor-Alpha (TNF-α), prostate-specific antigen (PSA), stem cell factor (SCF), RANTES, fractaline, among others as described above. Embodiment 17: The method of embodiment 16, wherein if the two or more immune proteins are increased compared to a predetermined threshold is indicative of vaginal dryness or vaginal irritation and the therapeutic solution comprises one or a combination of lubricants, moisturizers, or hormonal therapy.

Embodiment 18: The method of embodiment 1, wherein the therapeutic solution is selected from a group consisting of: hormonal therapies, antimicrobials, anti-inflammatory neuromodulators, vaginal moisturizers, antihistamines, or pelvic floor therapy.

Embodiment 19: The method of embodiment 1 further comprising administering a treatment to the patient

Embodiment 20: A method of treating vulvovaginal symptoms in a female patient in need thereof, the method comprising: a) determining the patient's levels of two or more biomarkers by: i) obtaining a biological sample from the patient, wherein the biological sample comprises a cervicovaginal lavage (CVL) sample or a vaginal swab; ii) measuring the levels of at least two or more biomarkers in the sample obtained in (i); and b) administering to the patient a treatment for the vulvovaginal symptoms if the levels of the at least two or more biomarkers deviate from a predetermined threshold.

Embodiment 21: The method of embodiment 20, wherein the vulvovaginal symptoms are caused by genitourinary syndrome of menopause (GSM). Embodiment 22: The method of embodiment 21, wherein the vulvovaginal symptoms comprise vaginal dryness, vaginal irritation, vaginal soreness, dyspareunia, vulvar dryness, vulvar irritation, vulvar soreness, and pain.

Embodiment 23: The method of embodiment 20, further comprising measuring the levels of at least five or more biomarkers. Embodiment 24: The method of embodiment 20, further comprising measuring the levels of at least ten or more biomarkers. Embodiment 25: The method of embodiment 20, further comprising measuring the levels of at least fifteen or more biomarkers.

Embodiment 26: The method of embodiment 20, wherein the two or more biomarkers comprise immune proteins, metabolites, bacterial abundance, or a combination thereof.

Embodiment 27: The method of embodiment 26, wherein the immune proteins comprise one or a combination of programmed cell death protein 1 (PD-1), programmed death-ligand 1 (PD-L1), interleukin-2 (IL-2), IL-7, alpha-fetoprotein (AFP), CYFRA 21-1 (cytokeratin 19 fragment), prostate-specific antigen (PSA), and/or carcinoembryonic antigen (CEA).

Embodiment 28: The method of embodiment 26, wherein the metabolites comprise one or more of: (i) lipids selected from glycerophospholipids, fatty acids, and sphingolipids; (ii) xenobiotics; or (iii) a combination thereof.

Embodiment 29: The method of embodiment 26 or embodiment 28, wherein the metabolites comprise one or a combination of laurylcarnitine (C12), 5-dodecenoylcarnitine, carnitine, myristoleoylcarnitine (C14:1), decanoylcarnitine (C10), mannonate, tricarballyate, citraconate/glutaconate, N-acetylglucosaminylasparagine, N-acetylglycine, N-acetylaspartate (NAA), N-acetylphenylalanine, N-delta-acetylornithine, N5-methyllysine, hydroxy-N6,N6,N6-trimethyllysine, N-acetylalanine, and gamma-glutamyl-epsilon-lysine, adenosine monophosphate (AMP) and 2′O-methylguanosine, p-cresol sulfate; and histamine.

Embodiment 30: The method of embodiment 28, wherein the lipids comprise one or a combination of behenoyl dihydrosphingomyelin (d18:0/22:0), sphingomyelin (d18:0/20:0, d16:0/22:0), 1-stearoyl-GPS (18:0), 1-palmitoyl-2-docosahexaenoyl-GPE (16:0/22:6), sphingomyelin (d18:1/22:1, d18:2/22:0, d16:1/24:1), sphingomyelin (d18:2/23:0, d18:1/23:1, d17:1/24:1), and sphingomyelin (d18:1/20:1, d18:2/20:0, d18:1/21:0, d17:1/22:0, d16:1/23:0).

Embodiment 31: The method of embodiment 26, wherein the bacterial abundance of Sneathia amnii, Megasphaera lornae, Group B Streptococcus, Lactobacillus crispatus, or a combination thereof, is measured.

Embodiment 32: An in vitro method of diagnosing and/or treating vulvovaginal symptoms in a female patient in need thereof, the method comprising: a) determining the patient's levels of two or more biomarkers by: i) obtaining a biological sample from the patient, wherein the biological sample comprises a cervicovaginal lavage (CVL) sample or a vaginal swab; ii) measuring the levels of at least two or more biomarkers in the sample obtained in (i); b) diagnosing the patient with vulvovaginal symptoms and administering a treatment when the levels of at least two biomarkers deviate from predetermined thresholds. In certain embodiments, the patient is diagnosed with vulvovaginal symptoms if the levels of at least two or more biomarkers deviate from a predetermined threshold.

Embodiment 33: The method of embodiment 32, wherein the vulvovaginal symptoms are caused by genitourinary syndrome of menopause (GSM). Embodiment 34: The method of embodiment 32, wherein the vulvovaginal symptoms comprise vaginal dryness, vaginal irritation, vaginal soreness, dyspareunia, vulvar dryness, vulvar irritation, vulvar soreness, and pain.

Embodiment 35: The method of embodiment 32, further comprising measuring the levels of at least five or more biomarkers. Embodiment 36: The method of embodiment 32, further comprising measuring the levels of at least ten or more biomarkers. Embodiment 37: The method of embodiment 32, further comprising measuring the levels of at least fifteen or more biomarkers.

Embodiment 38: The method of embodiment 32, wherein the two or more biomarkers comprise immune proteins, metabolites, bacterial abundance, or a combination thereof.

Embodiment 39: The method of embodiment 38, wherein the immune proteins comprise one or a combination of programmed cell death protein 1 (PD-1), programmed death-ligand 1 (PD-L1), interleukin-2 (IL-2), IL-7, alpha-fetoprotein (AFP), CYFRA 21-1 (cytokeratin 19 fragment), prostate-specific antigen (PSA), and/or carcinoembryonic antigen (CEA).

Embodiment 40: The method of embodiment 38, wherein the metabolites comprise one or more of: (i) lipids selected from glycerophospholipids, fatty acids, and sphingolipids; (ii) xenobiotics; or (iii) a combination thereof.

Embodiment 41: The method of embodiment 38 or embodiment 40, wherein the metabolites comprise one or a combination of laurylcarnitine (C12), 5-dodecenoylcarnitine, carnitine, myristoleoylcarnitine (C14:1), decanoylcarnitine (C10), mannonate, tricarballyate, citraconate/glutaconate, N-acetylglucosaminylasparagine, N-acetylglycine, N-acetylaspartate (NAA), N-acetylphenylalanine, N-delta-acetylornithine, N5-methyllysine, hydroxy-N6,N6,N6-trimethyllysine, N-acetylalanine, and gamma-glutamyl-epsilon-lysine, adenosine monophosphate (AMP) and 2′O-methylguanosine, p-cresol sulfate; and histamine.

Embodiment 42: The method of embodiment 38, wherein the lipids comprise one or a combination of behenoyl dihydrosphingomyelin (d18:0/22:0), sphingomyelin (d18:0/20:0, d16:0/22:0), 1-stearoyl-GPS (18:0), 1-palmitoyl-2-docosahexaenoyl-GPE (16:0/22:6), sphingomyelin (d18:1/22:1, d18:2/22:0, d16:1/24:1), sphingomyelin (d18:2/23:0, d18:1/23:1, d17:1/24:1), and sphingomyelin (d18:1/20:1, d18:2/20:0, d18:1/21:0, d17:1/22:0, d16:1/23:0).

Embodiment 43: The method of embodiment 38, wherein the bacterial abundance of Sneathia amnii, Megasphaera lornae, Group B Streptococcus, Lactobacillus crispatus, or a combination thereof, is measured.

Embodiment 44: The method of any one of embodiments 20-43, wherein the treatments selected from a group consisting of: hormonal therapies, antimicrobials, anti-inflammatories neuromodulators, vaginal moisturizers, antihistamines, or pelvic floor therapy.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method for providing a therapeutic solution to treat vulvovaginal symptoms in a female patient in need thereof, said method comprises:
a) determining the patient's level of two or more biomarkers by:
i) obtaining a biological sample from the patient, wherein the biological sample comprises a cervicovaginal lavage (CVL) sample or a vaginal swab; and
ii) measuring the levels of at least two or more biomarkers in the biological sample obtained in (i);
b) applying results from (a) to predetermined thresholds, wherein deviation of at least two biomarkers from the predetermined thresholds identifies at least one therapeutic solution for treating vulvovaginal symptoms; and
c) providing the therapeutic solution to a medical professional to determine treatment for the patient.

2. The method of claim 1, wherein the vulvovaginal symptoms are caused by genitourinary syndrome of menopause (GSM), wherein the vulvovaginal symptoms comprise vaginal dryness, vaginal irritation, vaginal soreness, dyspareunia, vulvar dryness, vulvar irritation, vulvar soreness, and pain.

3. The method of claim 1, wherein the two or more biomarkers comprise immune proteins, metabolites, bacterial abundance, or a combination thereof.

4. The method of claim 3, wherein the immune proteins comprise one or a combination of programmed cell death protein 1 (PD-1), programmed death-ligand 1 (PD-L1), interleukin-2 (IL-2), IL-7, alpha-fetoprotein (AFP), CYFRA 21-1 (cytokeratin 19 fragment), prostate-specific antigen (PSA), and/or carcinoembryonic antigen (CEA).

5. The method of claim 3, wherein the metabolites comprise one or more of: (i) lipids selected from glycerophospholipids, fatty acids, and sphingolipids; (ii) xenobiotics; or (iii) a combination thereof.

6. The method of claim 3, wherein the metabolites comprise one or a combination of laurylcarnitine (C12), 5-dodecenoylcarnitine, carnitine, myristoleoylcarnitine (C14:1), decanoylcarnitine (C10), mannonate, tricarballyate, citraconate/glutaconate, N-acetylglucosaminylasparagine, N-acetylglycine, N-acetylaspartate (NAA), N-acetylphenylalanine, N-delta-acetylornithine, N5-methyllysine, hydroxy-N6,N6,N6-trimethyllysine, N-acetylalanine, and gamma-glutamyl-epsilon-lysine, adenosine monophosphate (AMP) and 2′O-methylguanosine, p-cresol sulfate, histamine behenoyl dihydrosphingomyelin (d18:0/22:0), sphingomyelin (d18:0/20:0, d16:0/22:0), 1-stearoyl-GPS (18:0), 1-palmitoyl-2-docosahexaenoyl-GPE (16:0/22:6), sphingomyelin (d18:1/22:1, d18:2/22:0, d16:1/24:1), sphingomyelin (d18:2/23:0, d18:1/23:1, d17:1/24:1), and sphingomyelin (d18:1/20:1, d18:2/20:0, d18:1/21:0, d17:1/22:0, d16:1/23:0).

7. The method of claim 3, wherein the bacterial abundance of Sneathia amnii, Megasphaera lornae, Group B Streptococcus, Lactobacillus crispatus, or a combination thereof, is measured.

8. The method of claim 1, wherein at least one of the two or more biomarkers comprises histamine.

9. The method of claim 8, wherein the two or more biomarkers comprise histamine and at least one carnitine or a derivative thereof; wherein the at least one carnitine derivative is selected from a group consisting of laurylcarnitine (C12), 5-dodecenoylcarnitine, myristoleoyl-carnitine (C14:1), or decanoylcarnitine (C10).

10. The method of claim 9, wherein if histamine is increased compared to a predetermined threshold and the at least one carnitine or derivative thereof is decreased compared to a predetermined threshold then the therapeutic solution comprises an antihistamine.

11. The method of claim 1, wherein the two or more biomarkers comprises two or more immune proteins selected from a group consisting of programmed cell death protein 1 (PD-1), programmed death-ligand 1 (PD-L1), interleukin-2 (IL-2), IL-7, IL-12, IL-8, IL-10, Tumor Necrosis Factor-Alpha (TNF-α), prostate-specific antigen (PSA), stem cell factor (SCF), RANTES, or fractaline.

12. The method of claim 11, wherein if the two or more immune proteins are increased compared to a predetermined threshold is indicative of vaginal dryness or vaginal irritation and the therapeutic solution comprises one or a combination of lubricants, moisturizers, or hormonal therapy.

13. The method of claim 1, wherein the therapeutic solution is selected from a group consisting of: hormonal therapies, antimicrobials, anti-inflammatories, neuromodulators, vaginal moisturizers, antihistamines, or pelvic floor therapy.

14. The method of claim 1 further comprising administering a treatment to the patient.

15. A method of treating vulvovaginal symptoms in a female patient in need thereof, the method comprising:
a) determining the patient's levels of two or more biomarkers by:
i) obtaining a biological sample from the patient, wherein the biological sample comprises a cervicovaginal lavage (CVL) sample or a vaginal swab;
ii) measuring the levels of at least two or more biomarkers in the sample obtained in (i); and
b) administering to the patient a treatment for the vulvovaginal symptoms if the levels of the at least two or more biomarkers deviate from a predetermined threshold.

16. The method of claim 15, wherein the vulvovaginal symptoms are caused by genitourinary syndrome of menopause (GSM), wherein the vulvovaginal symptoms comprise vaginal dryness, vaginal irritation, vaginal soreness, dyspareunia, vulvar dryness, vulvar irritation, vulvar soreness, and pain.

17. The method of claim 15, wherein the two or more biomarkers comprise immune proteins, metabolites, bacterial abundance, or a combination thereof.

18. The method of claim 17, wherein the immune proteins comprise one or a combination of programmed cell death protein 1 (PD-1), programmed death-ligand 1 (PD-L1), interleukin-2 (IL-2), IL-7, alpha-fetoprotein (AFP), CYFRA 21-1 (cytokeratin 19 fragment), prostate-specific antigen (PSA), and/or carcinoembryonic antigen (CEA), wherein the metabolites comprise one or a combination of laurylcarnitine (C12), 5-dodecenoylcarnitine, carnitine, myristoleoylcarnitine (C14:1), decanoylcarnitine (C10), mannonate, tricarballyate, citraconate/glutaconate, N-acetylglucosaminylasparagine, N-acetylglycine, N-acetylaspartate (NAA), N-acetylphenylalanine, N-delta-acetylornithine, N5-methyllysine, hydroxy-N6,N6,N6-trimethyllysine, N-acetylalanine, and gamma-glutamyl-epsilon-lysine, adenosine monophosphate (AMP) and 2′O-methylguanosine, p-cresol sulfate, histamine behenoyl dihydrosphingomyelin (d18:0/22:0), sphingomyelin (d18:0/20:0, d16:0/22:0), 1-stearoyl-GPS (18:0), 1-palmitoyl-2-docosahexaenoyl-GPE (16:0/22:6), sphingomyelin (d18:1/22:1, d18:2/22:0, d16:1/24:1), sphingomyelin (d18:2/23:0, d18:1/23:1, d17:1/24:1), and sphingomyelin (d18:1/20:1, d18:2/20:0, d18:1/21:0, d17:1/22:0, d16:1/23:0), and wherein the bacterial abundance of Sneathia amnii, Megasphaera lornae, Group B Streptococcus, Lactobacillus crispatus, or a combination thereof, is measured.

19. The method of claim 15, wherein the treatment is selected from a group consisting of: hormonal therapies, antimicrobials, anti-inflammatories, neuromodulators, vaginal moisturizers, antihistamines, or pelvic floor therapy.
